(19) United States
(12) Patent Application Publication
(10) Pub. No.: US 2011/0015162 A1
(43) Pub. Date: Jan. 20, 2011

(54) 15,16-Methylene-17-hydroxy-19-nor-21-carboxylic acid-steroid Y-lactone derivative, use thereof and medicinal products containing the derivative

(75) Inventors: Ulrich Klar, Berlin (DE); Joachim Kuhnke, Potsdam (DE); Rolf Bohlmann, Berlin (DE); Jan Huebner, Berlin (DE); Sven Ring, Jena (DE); Thomas Frenzel, Hofheim (DE); Frederik Menges, Schriesheim (DE); Steffen Borden, Berlin (DE); Hans Peter Muhn, Berlin (DE); Katja Prelle, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Bayer Schering Pharma Aktiengesellschaft, Berlin (DE)

(21) Appl. No.: 12/811,002

(22) PCT Filed: Dec. 23, 2008
(86) PCT No.: PCT/EP08/11162

§ 371 Date: Oct. 4, 2010

(30) Foreign Application Priority Data
Dec. 29, 2007 (DE) 10 2007 063 501.1

(51) Int. Cl. A61K 31/58; C07J 53/00; A61K 31/56

(52) U.S. Cl. 514/170; 540/15; 514/173

(57) ABSTRACT

The invention relates to 15,16-methylene-17-hydroxy-19-nor-21-carboxylic acid-steroid γ-lactone derivatives with the general chemical formula I, and R⁴, R^{6a}, R^{6b}, R⁷ and Z have the meanings stated in claim 1, and their solvates, hydrates, stereoisomers and salts. The invention further relates to the use of these derivatives for the production of a medicinal product for oral contraception and for the treatment of pre-, peri- and postmenopausal complaints and medicinal products that contain said derivatives. The derivatives according to the invention have a progestational and in preferred cases additionally an antimineralocorticoid and neutral to slightly androgenic action.

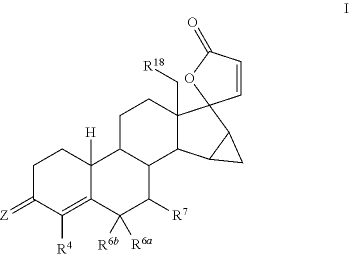

The invention relates to 15,16-methylene-17-hydroxy-19-nor-carboxylic acid-steroid γ-lactone derivatives with α,β-unsaturated 17,17-spirolactone, use thereof and medicinal products containing the derivatives with progestational action, for example for the treatment of pre-, peri- and postmenopausal and of premenstrual complaints.

Compounds with progestational, antimineralocorticoid, antiandrogenic or antiestrogenic action based on a steroid structure are known from the literature, derived for example from 19-nor-androst-4-en-3-one or a derivative thereof (the numbering of the steroid structure is given for example in Fresenius/Görlitzer 3rd Ed. 1991 “Organic-Chemical Nomenclature” p. 60 ff.).

Thus, WO 2006072467 A1 discloses the compound 6β, 7β;15β,16β-dimethylene-3-oxo-17-pregn-4-ene-21,17β-carbolactone (drospirenone), which has progestational action and has been used for example in an oral contraceptive and in a preparation for the treatment of postmenopausal complaints. Owing to its comparatively low affinity for the progestogen receptor and its comparatively high ovulation-inhibiting dose, however, drospirenone is contained in the contraceptive at the relatively high daily dose of 3 mg. Drospirenone is, moreover, also characterized in that in addition to the progestational action it also has aldosterone-antagonistic (antimineralocorticoid) and antiandrogenic action. These two properties make drospirenone very similar in its pharmacological profile to the natural progestogen, progesterone, which however, unlike drospirenone, is not sufficiently bioavailable orally. In order to lower the dose to be administered, WO 2006072467 A1 further proposes an 18-methyl-19-nor-17-pregn-4-ene-21,17-carbolactone and pharmaceutical preparations containing this, which have a higher progestational potency than drospirenone.

In addition, U.S. Pat. No. 3,705,179, for example, discloses steroids that display antiandrogenic activity and are suitable for the treatment of diseases that are linked to androgens.

Furthermore, U.S. Pat. No. 2,918,463 discloses 17-carboxyalkylated 17-hydroxy-19-nor-androsten-3-ones, including 17α-(2-carboxyvinyl)-17β-hydroxy-19-nor-androst-4-en-3-one lactone. The compounds described are said to block the action of deoxycorticosterone acetate on the level of sodium and potassium in the urine and simultaneously, at higher concentration, have a salt-binding action. Moreover, these compounds are also said to be effective against hypertension.

The aim of the present invention is to make compounds available that bind strongly to the progestogen receptor. Moreover, the compounds should preferably also have antimineralocorticoid action and, with respect to the androgen receptor, a neutral to slightly androgenic action. Another essential aim of the present invention consists of achieving a balanced action profile with respect to the progestational action to the antimineralocorticoid action, so that the ratio of the progestational action to the antimineralocorticoid action is less than with drospirenone.

This aim is achieved with the 15,16-methylene-17-hydroxy-19-nor-carboxylic acid-steroid γ-lactone derivatives according to the invention according to claim 1, the use of the derivatives according to the invention according to claim 12 and a medicinal product containing at least one derivative according to the invention according to claim 14. Advantageous embodiments of the invention are stated in the subclaims.

The present invention accordingly relates to 15,16-methylene-17-hydroxy-19-nor-carboxylic acid-steroid γ-lactone derivatives with the general chemical formula I,

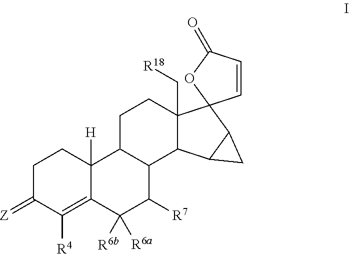

in which
Z is selected from the group comprising oxygen, two hydrogen atoms, NOR′ and NNHSO₂R′, in which R′ is hydrogen, C₁-C₁₀-alkyl, aryl or C₇-C₂₀-aralkyl,
R⁴ is selected from the group comprising hydrogen and halogen,

furthermore either:
R^{6a}, R^{6b} in each case independently of one another are selected from the group comprising hydrogen, C₁-C₁₀-alkyl, C₂-C₁₀-alkenyl and C₂-C₁₀-alkynyl, or together form methylene or 1,2-ethanediyl and
R⁷ is selected from the group comprising hydrogen, C₁-C₁₀-alkyl, C₃-C₆-cycloalkyl, C₂-C₁₀-alkenyl and C₂-C₁₀-alkynyl,

or:
R^{6a} together form an oxygen atom or methylene or drop out with formation of a double bond between C⁶ and C⁷ and
R^{6b} is selected from the group comprising hydrogen, C₁-C₁₀-alkyl, C₂-C₁₀-alkenyl and C₂-C₁₀-alkynyl and
R¹⁸ is hydrogen or C₁-C₃-alkyl,
and their solvates, hydrates, stereoisomers and salts.

The numbering of the carbon backbone of the derivative according to the invention with the general chemical formula I follows the numbering of a steroid structure in the usual way, as described for example in Fresenius, loc. cit. The numbering of the residues stated in the claims corresponds in a similar manner to their bonding position on the carbon backbone of the derivative, as far as this relates to R⁴, R⁶, R⁷ and R¹⁸. For example, the residue R⁴ binds to the C⁴-position of the derivative according to the invention.

With respect to the groups defined for Z, the groups NOR′ and NNHSO₂R′ each bind with a double bond via N to the carbon backbone of the derivative according to —NOR′ or ═NNH—SO₂R′. OR in NOR′ and NHSO₂R′ in NNHSO₂R′ can be in syn- or anti-position.

Alkyl in R′, R^{6a}, R^{6b} and R⁷ and in R¹⁹, R²⁰, R^{21a}, R^{21b} and R²² means, in the general chemical formulae given later, linear or branched alkyl groups with 1-10 carbon atoms, for example methyl, ethyl, propyl, isopropyl, butyl, isobutyl, tert.-butyl, pentyl, isopentyl, neopentyl, heptyl, hexyl, decyl. Alkyl in R¹⁸ means methyl, ethyl, propyl or isopropyl. The alkyl groups R′, R^{6a}, R^{6b}, R⁷, R¹⁸, R¹⁹, R²⁰, R²³, R^{21b} and R²² can moreover be perfluorinated or can be substituted with 1-5 halogen atoms, hydroxyl groups, C₁-C₄-alkoxy groups, C₆-C₁₂-aryl groups (which in their turn can be substituted with 1-3 halogen atoms). In particular, alkyl can therefore also stand for hydroxymethylene (HO—CH₂), hydroxyethylene (HO—C₂H₄), hydroxypropylene (HO—C₃H₆) and hydroxybutylene (HO—C₄H₈) and their isomers.

Alkenyl in R^{6a}, R^{6b} and R⁷ means linear or branched alkenyl groups with 2-10 carbon atoms, for example vinyl, propenyl, butenyl, pentenyl, isobutenyl, isopentenyl.

Alkynyl in R^{6a}, R^{6b} and R⁷ means linear or branched alkynyl groups with 2-10 carbon atoms, for example ethynyl, propynyl, butynyl, pentynyl, isobutynyl, isopentynyl.

The alkenyl and alkynyl groups R^{6a}, R^{6a} and R⁷ can be substituted with 1-5 halogen atoms, hydroxyl groups, C₁-C₃-alkoxy groups, C₆-C₁₂-aryl groups (which in their turn can be substituted with 1-3 halogen atoms).

Cycloalkyl in R⁷ means cycloalkyl groups with 3-6 carbon atoms, for example cyclopropyl, cyclobutyl, cyclopentyl and cyclohexyl. The cycloalkyl groups R⁷ can be substituted with halogen, OH, O-alkyl, CO₂H, CO₂-alkyl, NH₂, NO₂, N₃, CN, C₁-C₁₀-alkyl, C₁-C₁₀-acyl, C₁-C₁₀-acyloxy groups.

Aryl in R′ means substituted and unsubstituted carbocyclic or heterocyclic residues with one or more heteroatoms, e.g. phenyl, naphthyl, furyl, thienyl, pyridyl, pyrazolyl, pyrimidinyl, oxazolyl, pyridazinyl, pyrazinyl, quinolyl, thiazolyl, which can be substituted singly or multiply with halogen, OH, O-alkyl, CO₂H, CO₂-alkyl, NH₂, NO₂, N₃, CN, C₁-C₁₀-alkyl, C₁-C₁₀-acyl, C₁-C₁₀-acyloxy groups. If aryl is otherwise mentioned as substituent on alkyl, alkenyl or alkynyl, this refers in particular to aryl groups with 6-12 ring carbon atoms.

Aralkyl in R′ and R⁷ means aralkyl groups that can contain up to 14 carbon atoms, preferably 6 to 10 carbon atoms, in the ring, and 1 to 8, preferably 1 to 4, carbon atoms in the alkyl chain. As aralkyl residues, consideration can be given for example to benzyl, phenylethyl, naphthylmethyl, naphthylethyl, furylmethyl, thienylethyl, pyridylpropyl. The rings can be substituted singly or multiply with halogen, OH, O-alkyl, CO₂H, CO₂-alkyl, NO₂, N₃, CN, C₁-C₂₀-alkyl, C₁-C₂₀-acyl, acyloxy groups.

If alkoxy (O-alkyl) is mentioned as substituent on alkyl, this refers to alkoxy groups with 1-4 carbon atoms, and if alkoxy is mentioned as substituent on alkenyl and alkynyl, this refers to alkoxy groups with 1-3 carbon atoms. Alkoxy can in particular be methoxy, ethoxy and propoxy.

If acyl (CO-alkyl) is mentioned as substituent on cycloalkyl and aryl, this refers to acyl groups with 1-carbon atoms, and if acyl is mentioned as substituent on aralkyl, this refers to acyl groups with 1-20 carbon atoms. Acyl can in particular be formyl, acetyl, propionyl and butyryl.

If acyloxy (O—CO-alkyl) is mentioned as substituent on cycloalkyl and aryl, this refers to acyloxy groups with 1-10 carbon atoms, and if acyloxy is mentioned as substituent on aralkyl, this refers to acyloxy groups with 1-20 carbon atoms. Acyloxy can in particular be formyloxy, acetyloxy, propionyloxy and butyryloxy.

Halogen means fluorine, chlorine or bromine.

According to a preferred embodiment of the invention, Z is selected from the group comprising oxygen, NOR′ and NNHSO₂R′.

According to another preferred embodiment of the invention, Z stands for oxygen.

According to another preferred embodiment of the invention, R⁴ is hydrogen or chlorine.

According to another preferred embodiment of the invention, R^{6a}, R^{6b} together form 1,2-ethanediyl or are each hydrogen.

According to another preferred embodiment of the invention, R⁷ is selected from the group comprising hydrogen, methyl, ethyl and vinyl.

According to another preferred embodiment of the invention, R⁶% R⁷ together form methylene.

According to another preferred embodiment of the invention, R^{7a} and R⁷ drop out with formation of a double bond between C⁶ and C⁷.

According to another preferred embodiment of the invention, R¹⁸ is hydrogen or methyl.

Compounds with the chemical formula I are preferred, in which
Z is oxygen, a group NOR′, where R′ is hydrogen, C₁-C₆-alkyl, aryl or C₇-C₁₂-aralkyl,
R⁴ is hydrogen or halogen,
   and either:
R^{6a}, R^{6b} independently of one another, are hydrogen, C₁-C₆-alkyl, C₂-C₆-alkenyl or C₂-C₆-alkynyl or together form methylene or 1,2-ethanediyl and
R⁷ is hydrogen, C₁-C₆-alkyl, C₃-C₆-cycloalkyl, C₂-C₆alkenyl or C₂-C₆-alkynyl,
   or:
R^{6a}, R⁷ drop out with formation of a double bond between C⁶ and C⁷ or together form methylene and
R^{6b} is selected from the group comprising hydrogen, C₁-C₆-alkyl, C₂-C₆-alkenyl and C₂-C₆-alkynyl,
R¹⁸ is hydrogen, methyl or ethyl.

Compounds with the formula I are especially preferred in which
Z is oxygen or a group NOR′, where R′ is hydrogen or C₁-C₃-alkyl,
R⁴ is hydrogen, chlorine or bromine,
   and either:
R^{6a}, R^{6b} independently of one another, are hydrogen, C₁-C₃-alkyl or C₂-C₄-alkenyl or together form methylene or 1,2-ethanediyl and
R⁷ is hydrogen, C₁-C₄-alkyl, C₃-C₄-cycloalkyl or C₂-C₄-alkenyl,
   or:
R^{6a}, R⁷ drop out with formation of a double bond between C⁶ and C⁷ or together form methylene and
R^{6a}, R⁷ drop out with formation of a double bond between C⁶ and C⁷ or together form methylene and
R¹⁸ is hydrogen or methyl.

All possible stereoisomers and isomeric mixtures, including racemates, of the compound with the general chemical formula I are hereby expressly included, and moreover the position of the unsaturated γ-lactone ring in the derivative according to the invention can also occur in two isomeric forms. Each of the stated substituents on the steroid basic structure can be both in an α position and in a β position. Furthermore, the substituents on the steroid basic structure that contain a double bond and in which the double bond to each carbon atom carries at least one substituent, which is not hydrogen, can be both E- and Z-configured. Groups bound to two adjacent carbon atoms of the structure, for example an oxygen atom, methylene or 1,2-ethanediyl, are bound either in α,α-position or in β,β-position.

All crystal modifications of the compound with the general chemical formula I are also expressly included.

Derivatives according to the invention in the form of solvates, in particular of hydrates, are also expressly included, and the compounds according to the invention can accordingly contain polar solvents, in particular water, as structural element of the crystal lattice of the compounds according to the invention. The polar solvent, in particular water, can be present in stoichiometric proportions or even in nonstoichiometric proportions. Stoichiometric solvates and hydrates are also called hemi-, (semi-), mono-, sesqui-, di-, tri-, tetra-, penta-, etc. solvates or hydrates.

If an acid function is present, the physiologically compatible salts of organic and inorganic bases are suitable as salts, for example the readily soluble alkali-metal and alkaline-earth salts, and the salts of N-methyl-glucamine, D-methyl-glucamine, ethyl-glucamine, lysine, 1,6-hexadiamine, ethanolamine, glucosamine, sarcosine, serinol, Tris-hydroxy-methyl-aminomethane, aminopropanediol, Sovak-base, 1-amino-2,3,4-butanetriol. If a basic function is present, the physiologically compatible salts of organic and inorganic acids are suitable, such as of hydrochloric acid, sulfuric acid, phosphoric acid, citric acid, tartaric acid etc.

It was found that the compounds or derivatives according to the invention have good progestational action. Furthermore, some interesting compounds according to the invention interact with the mineralocorticoid receptor and are able to impart an antagonistic action. Moreover, the compounds according to the invention have a neutral to slightly androgenic action with respect to the androgen receptor. Another property of the majority of the compounds is that the bonds of these compounds to the progesterone receptor and to the mineralocorticoid receptor are balanced relative to one another, namely so that their ratio of the capacity for binding to the progesterone receptor to the capacity for binding to the mineralocorticoid receptor is less than in the case of drospirenone. Therefore the antimineralocorticoid action of these compounds at given progestational action is less than with drospirenone. If the dosage of a given compound according to the invention is based on its progestational action, the antimineralocorticoid action of this compound at this dosage is therefore less than with drospirenone.

The compounds listed below are preferred according to the invention:

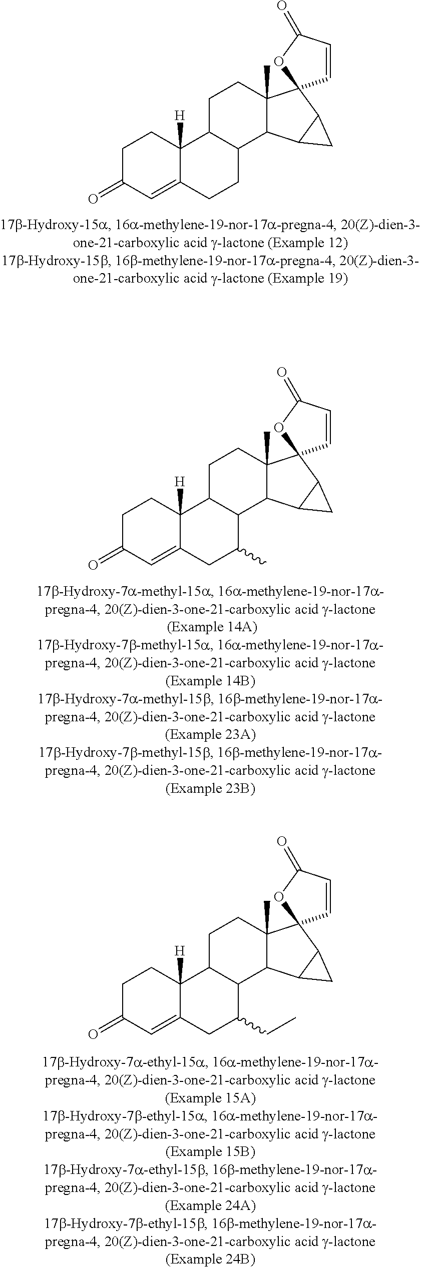

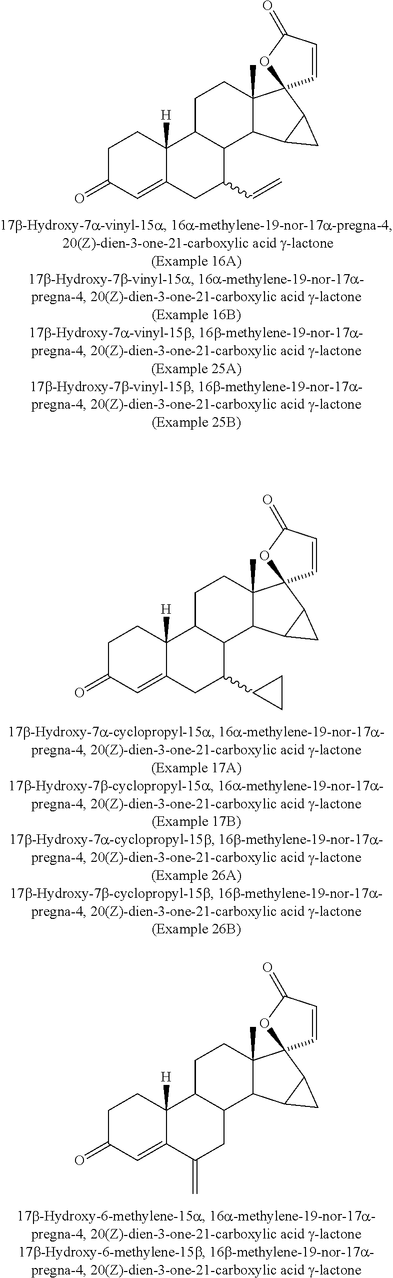

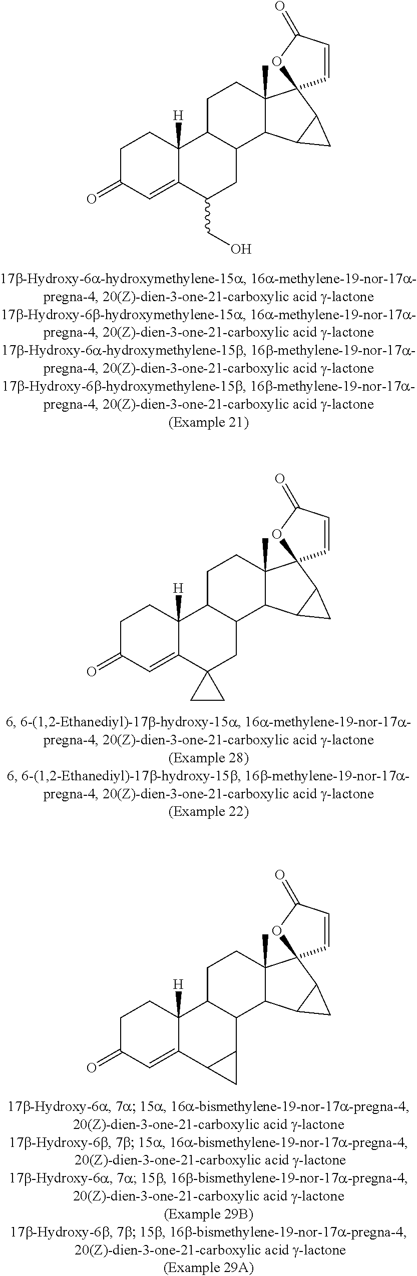

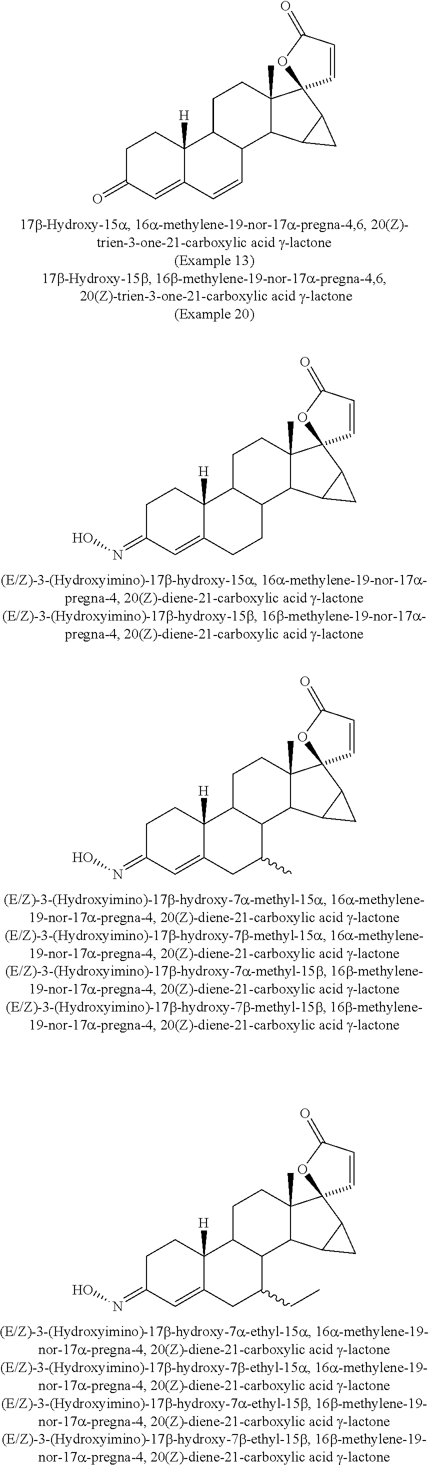

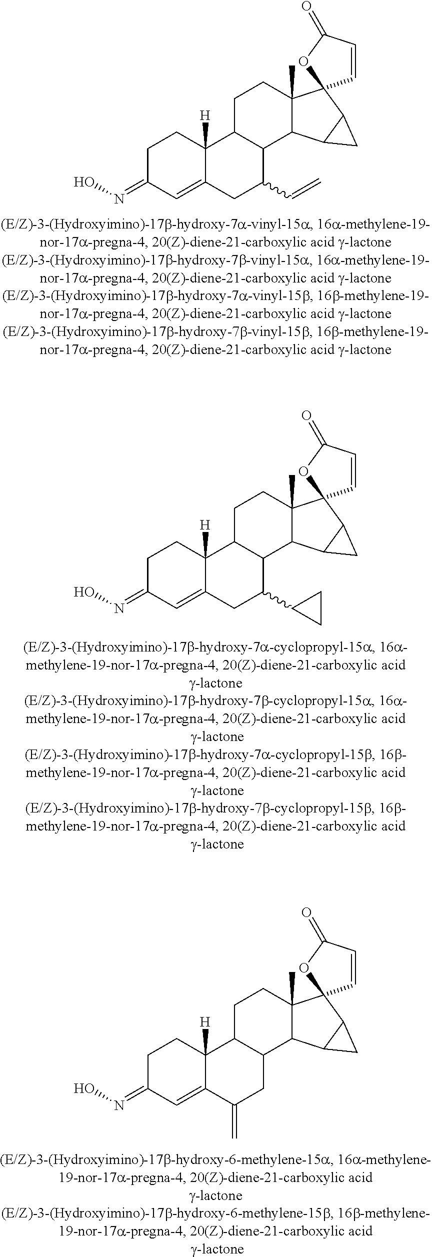

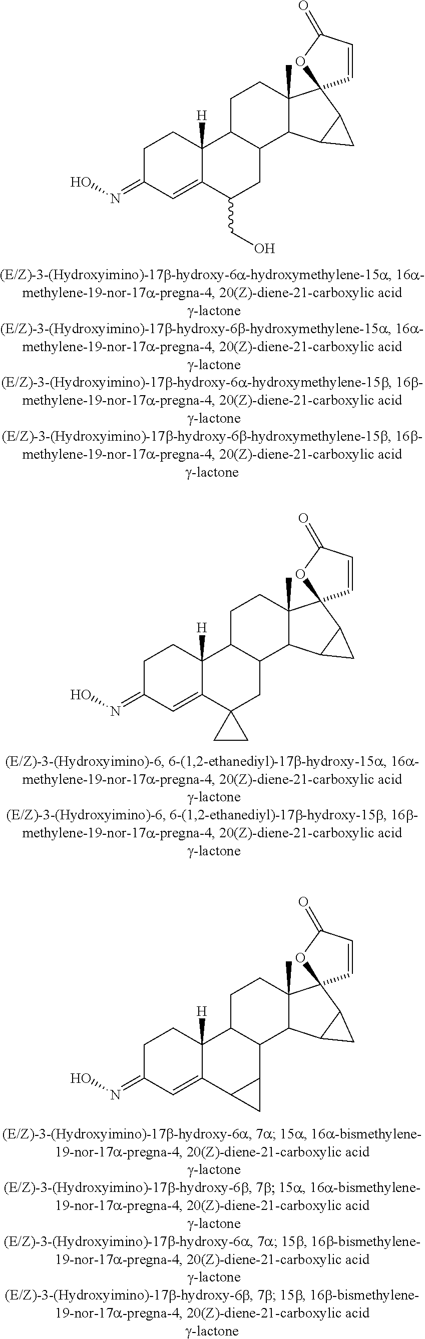

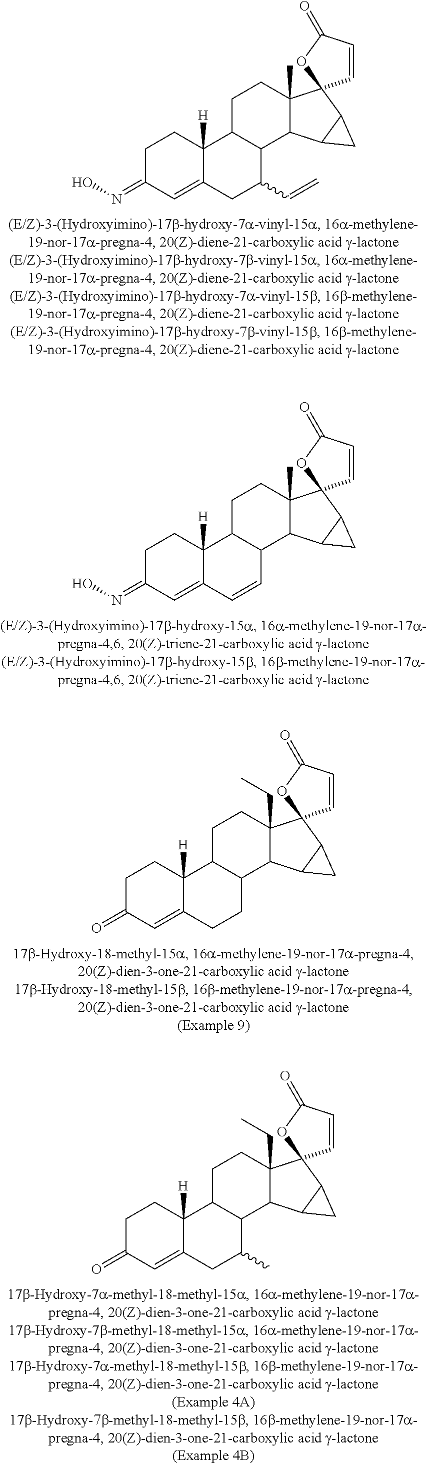

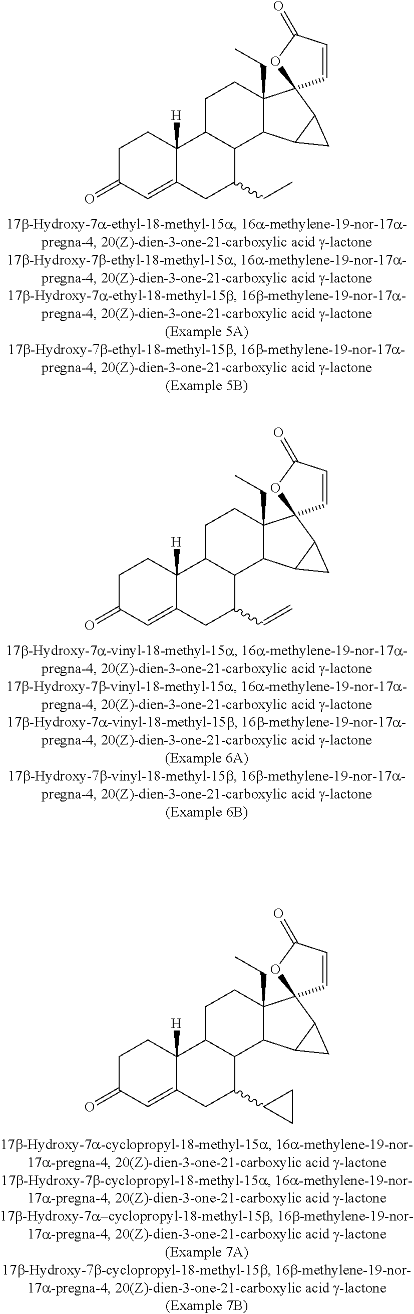

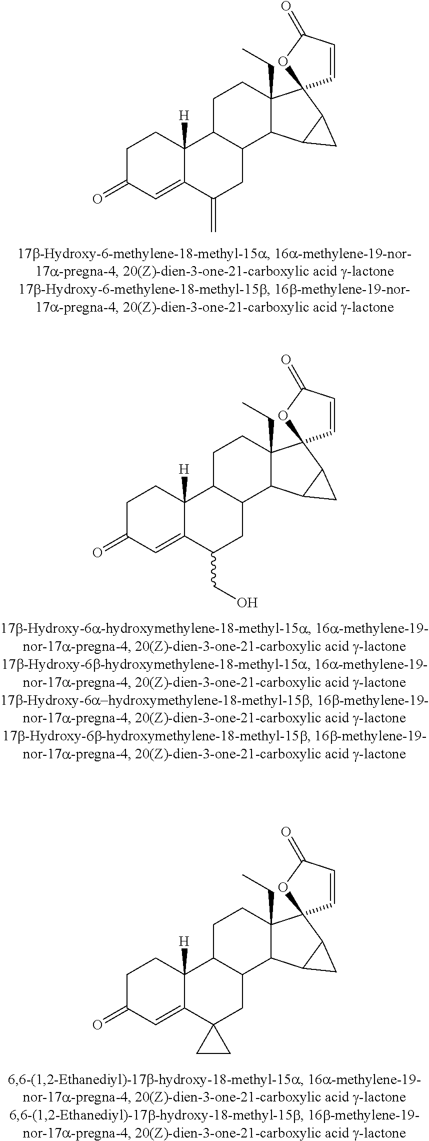

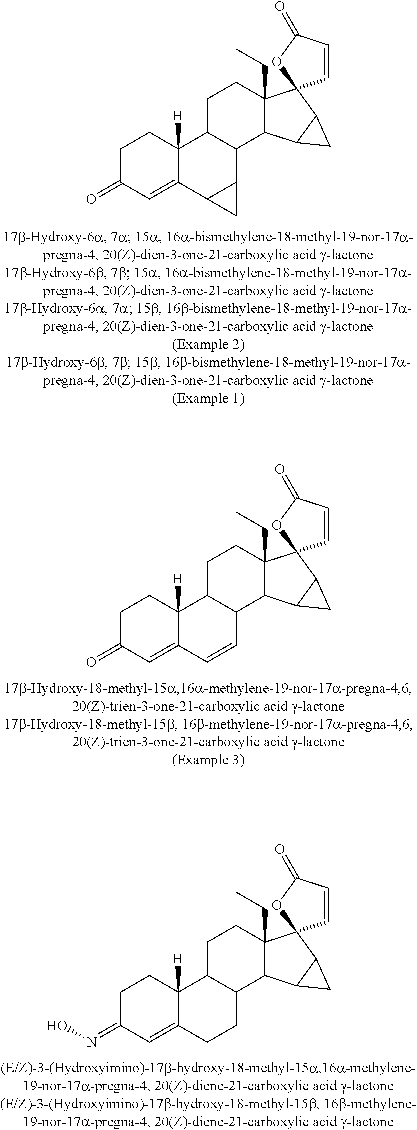

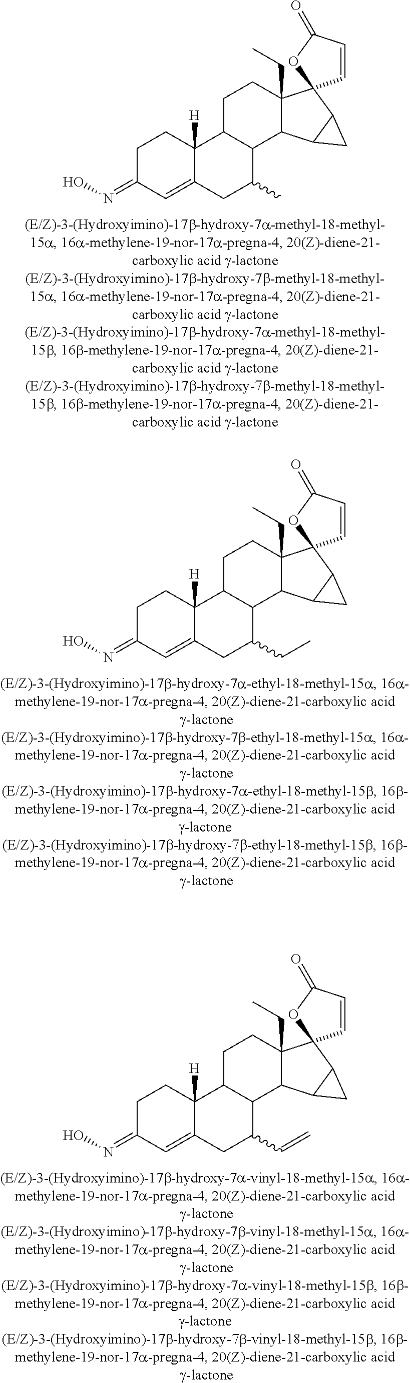

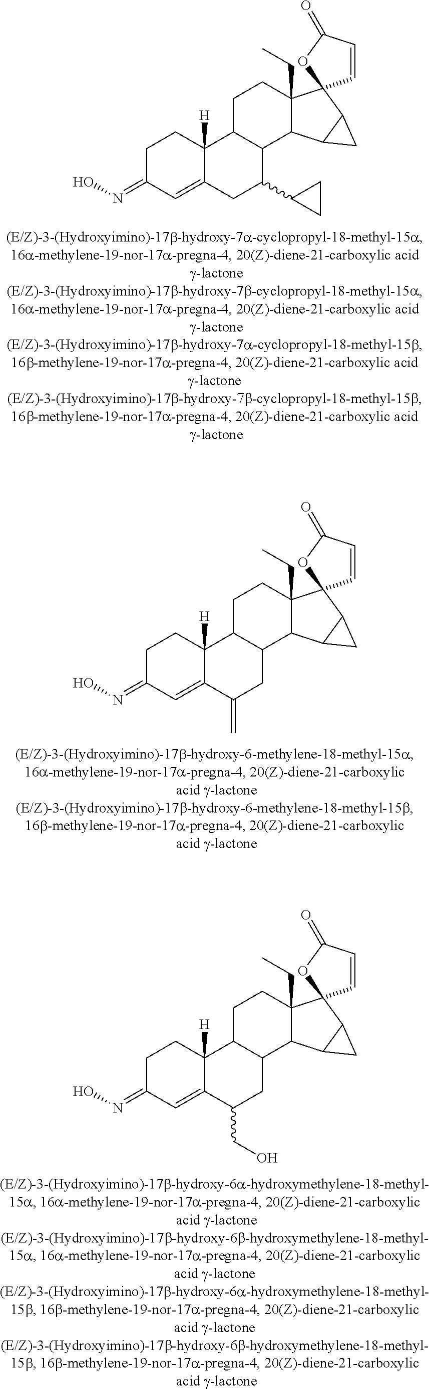

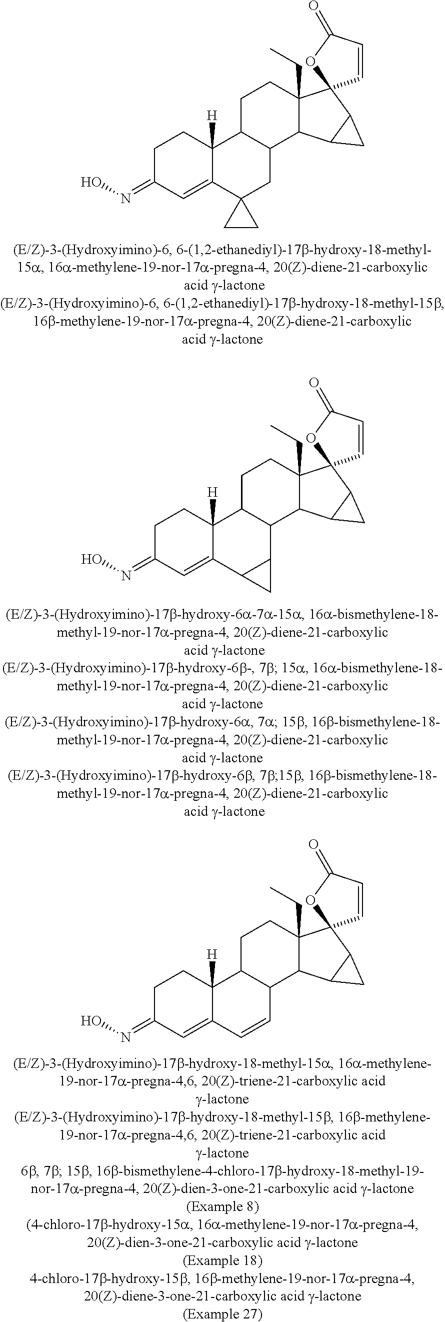

On the basis of their progestational efficacy, the novel compounds with the general chemical formula I can be used alone or in combination with estrogen in medicinal products for contraception.

The derivatives according to the invention are therefore suitable in particular for the production of a medicinal product for oral contraception and for the treatment of pre-, peri- and postmenopausal complaints, including use in preparations for hormone replacement therapy (HRT).

Owing to their favorable action profile, the derivatives according to the invention are moreover especially well suited to the treatment of premenstrual complaints, such as headaches, depressive moods, water retention and mastodynia.

The use of the derivatives according to the invention is especially preferred for the production of a medicinal product with progestational, and preferably also antimineralocorticoid and neutral to slightly androgenic action.

Treatment with the derivatives according to the invention is preferably applied to humans, but can also be carried out on related mammalian species, for example dog and cat.

For use of the derivatives according to the invention as medicinal products, they are combined with at least one suitable pharmaceutically harmless additive, for example a carrier. The additive is for example suitable for parenteral, preferably oral, application. Relevant materials are pharmaceutically suitable organic or inorganic inert additives, for example water, gelatin, gum arabic, lactose, starch, magnesium stearate, talc, vegetable oils, polyalkylene glycols etc. The medicinal products can be in solid form, for example as tablets, coated tables, suppositories, capsules, or in liquid form, for example as solutions, suspensions or emulsions. Optionally they also contain excipients, such as preservatives, stabilizers, wetting agents or emulsifiers, salts for altering the osmotic pressure or buffers. For parenteral application, oily solutions are suitable in particular, for example solutions in sesame oil, castor oil and, cottonseed oil are suitable. To increase the solubility, solubilizers can be added, for example benzyl benzoate or benzyl alcohol. It is also possible to incorporate the derivatives according to the invention in a transdermal system and therefore apply them transdermally. For oral application, consideration may be given in particular to tablets, coated tablets, capsules, pills, suspensions or solutions.

Further examples of administration routes are intravaginal or intrauterine administration. This is possible with physiologically tolerated solutions such as, for example, an aqueous or oily solution with or without suitable solubilizers, dispersants or emulsifiers. Examples of suitable oils are peanut oil, cottonseed oil, castor oil or sesame oil. The selection is by no means restricted thereto. For intravaginal or intrauterine administration it is possible to use special systems such as an intravaginal system (e.g. vaginal ring, VRS) or an intrauterine system (IUS) which release an active substance of the present invention from a reservoir over a prolonged period (e.g. 1, 2, 3, 4 or 5 years).

A representative example of an intrauterine system which may be mentioned is MIRENA®. This is a T-shaped, levonorgestrel-releasing intrauterine system from Bayer Schering Pharma AG.

Administration is further possible via an implanted depot system composed of an inert carrier material such as, for example, a biodegradable polymer or a synthetic silicone polymer. These depot systems release the active ingredient in a controlled manner over a prolonged period (e.g. 3 months to 3 years) and are implanted subcutaneously.

The dosage, of the derivatives according to the invention in contraceptive preparations should be 0.01 to 10 mg per day. The daily dose in the treatment of premenstrual complaints is around 0.1 to 20 mg. The progestational derivatives according to the invention in contraceptive preparations and in medicinal products for the treatment of premenstrual complaints are preferably administered orally. The daily dose is preferably administered as a single dose. The aforementioned dosages relate to oral administration forms.

On use of a depot formulation, the appropriate dosage, equivalent to the aforementioned oral dosages, is released continuously each day from the depot systems described above and employed in the long term.

A depot formulation, for example an IUS, releases per day an amount of 0.005 to 10 mg of a compound of general formula 1.

The progestational and estrogenic active components are preferably applied together orally in contraceptive preparations. The daily dose is preferably administered as a single dose.

As estrogens, consideration may be given to synthetic estrogens, preferably ethinylestradiol, but also mestranol, and natural estrogens, including phytoestrogens.

The estrogen is administered in a daily amount that corresponds to the pharmacological action of 0.01 to 0.04 mg ethinylestradiol. This amount relates to an oral administration form. If a different administration route is chosen, an appropriate dosage amount equivalent to the aforementioned oral dosage is to be used.

As estrogens in medicinal products for the treatment of pre-, peri- and postmenopausal complaints and for hormone replacement therapy, natural estrogens are mainly used, in particular estradiol, but also the esters of estradiol, for example estradiol valerate, or, also conjugated estrogens (CEEs=conjugated equine estrogens).

The progestational, antimineralocorticoid and androgenic or antiandrogenic action of the compounds according to the invention was investigated by the following methods:

### 1. Progesterone Receptor Binding Test:

Using cytosol from progesterone receptor-expressing insect cells (Hi5), competitive binding to the progesterone receptor was determined from the ability to displace ³H-progesterone as reference substance from the receptor. If a compound has an affinity corresponding to progesterone, this corresponds to a competition factor (CF) of 1, CF values greater than 1 are characterized by a lower affinity for the progesterone receptor, and CF values of less than 1 are characterized by higher affinity.

### 2. Mineralocorticoid Receptor Binding Test:

The test was carried out as in 1, with the following modifications: cytosol from mineralocorticoid receptor-expressing insect cells (Hi5) was used, and the reference substance was ³H-aldosterone.

### 3. Androgen Receptor Binding Test:

The test was carried out as in 1, with the following modifications: cytosol from androgen receptor-expressing insect cells (Hi5) was used, and the reference substance was ³H-testosterone.

The results of the binding tests and the ratio of the competition factors CF(PR) and CR(MR) are shown in Table 1, which for comparison also shows receptor binding values of drospirenone as reference substance A.

### 4. Determination of Progestational Action by Means of Transactivation Tests:

The culture medium used for culture of the cells used for the assay was DMEM (Dulbecco's Modified Eagle Medium: 4500 mg/ml glucose; PAA, #E15-009) with 10% FCS (Biochrom, S0115, batch #615B), 4 mM L-glutamine, 1% penicillin/streptomycin, 1 mg/ml G418 and 0.5 μg/ml puromycin.

Reporter cell lines (CHO K1 cells stably transfected with a fusion protein from the PR-ligand-binding domain and a Gal4-transactivation domain and a reporter construct, which contained luciferase under the control of a Gal4-responsive promoter) were seeded at a density of 4×10⁴ cells per well in white, opaque tissue culture plates each with 96 wells (PerkinElmer, #P12-106-017) and kept in culture medium with 3% DCC-FCS (serum treated with activated charcoal to remove interfering components contained in the serum). The test compounds were added eight hours later, and the cells were incubated with the compounds for 16 hours. The tests were carried out in triplicate. At the end of incubation the medium containing the effector was removed and replaced with lysis buffer. After luciferase assay substrate (Promega, #E1501) had been added, the 96-well plates were then put in a microplate luminometer (Pherastar, BMG Labtech), and the luminescence was measured. The IC₅₀ values were evaluated using software for calculating dose-effect relations. Table 2 presents the test results and, for comparison, corresponding results for drospirenone as reference substance A.

### 5. Determination of Antimineralocorticoid Action by Means of Transactivation Tests:

The antimineralocorticoid activity of the test substances was determined as for the transactivation tests described above.

The following modifications were undertaken: In this case reporter cell lines were used (MDCK cells) that express the human mineralocorticoid receptor, and transiently contain a reporter construct that contains luciferase under the control of a steroid hormone-responsive promoter.

The culture medium used for cultivation of the cells used for the assay was DMEM EARLE'S MEM (PAA, Cat.: E15-025) provided with 100U penicillin/0.1 mg/ml streptomycin (PAA, Cat: P11-010), 4 mM L-glutamine (PAA, Cat: M11-004) and fetal calf serum (BIO Witthaker, Cat: DE14-801F).

For determination of antimineralocorticoid efficacy, 1 nM aldosterone (SIGMA A-6628, Lot 22H4033) was added to the cells, to achieve almost maximum stimulation of the reporter gene. Inhibition of the effect indicated a mineralocorticoid-antagonistic action of the substances (Table 2; for comparison, corresponding values for drospirenone (A)).

### 6. Determination of Androgenic/Antiandrogenic Action by Means of Transactivation Tests:

The androgenic/antiandrogenic action of the test substances was determined as for the transactivation tests described above.

The following modifications were made: In this case reporter cell lines were used (PC3 cells) that express the androgen receptor, and a reporter construct that contains luciferase under the control of a steroid hormone-responsive promoter.

The culture medium used for cultivation of the cells used for the assay was RPMI medium without phenol red (PAA, #E15-49), provided with 100U penicillin/0.1 mg/ml streptomycin (PAA, Cat: P11-010), 4 mM L-glutamine (PAA, Cat: M11-004) and fetal calf serum (BIO Witthaker, Cat: DE14-801F).

For determination of the antiandrogenic efficacy, 0.05 nM R1881 was added to the cells, in order to achieve almost maximum stimulation of the reporter gene. Inhibition of the effect indicated an androgen-antagonistic action of the substances (Table 2; for comparison, corresponding values for drospirenone (A)).

If the production of the starting compounds is not described here, these are known to a person skilled in the art or can be prepared similarly to known compounds or methods described here. The isomeric mixtures can be separated into the individual compounds by the usual methods, for example crystallization, chromatography or salt formation. The salts are prepared in the usual way, by adding, to a solution of the compound with the general chemical formula I, the equivalent amount or an excess of a base or acid, which is optionally in solution, if necessary separating the precipitate or processing the solution in the usual way.

The compounds with the general chemical formula I are prepared, starting from compounds with the general chemical formula 1 (Scheme 2), according to the method shown in Scheme 1, in which R⁴, R^{6a}, R^{6b}, R⁷, R¹⁸ and Z have the meanings stated previously and in which
R⁶, R⁷ in 5 and 6, together, are an oxygen atom or a methylene group,
U is an oxygen atom, two alkoxy groups OR¹⁹, a C₂-C₁₀-alkylene-α,ω-dioxy group, which can be linear or branched, and R¹⁹ stands for a C₁-C₂₀-alkyl residue,
R²⁰ is a C₁-C₂₀-alkyl residue,
X is an NR^{21a}R^{21b}) group, an alkoxy group OR²²,
R^{21a}, R^{21b}) independently of one another, are hydrogen, C₁-C₁₀-alkyl or together form a C₄-C₁₀-α,ω-alkylene group, which can be linear or branched,
R²² is a C₁-C₂₀-alkyl residue.

Compounds 2 and 3 in Scheme 1 each have a double bond between C⁵ and C⁶ or between C⁵ and C¹⁰ and another double bond between C² and C³ or between C³ and C⁴.

Compounds 7 to 9 in Scheme 1 each have a double bond between C⁴ and C⁵ or between C⁵ and C⁶ or between C⁵ and C¹⁰.

For a person skilled in the art it is obvious that in the descriptions of the synthetic transformations it is always assumed that if necessary other functional groups present on the steroid structure are suitably protected.

The introduction of a 6,7-double bond with formation of compounds with the general chemical formulae 4, 13 or 18 is carried out by bromination of the respective 3,5-dienol ethers 3, 12 or 17 followed by elimination of hydrogen bromide (see for example J. Fried, J. A. Edwards, Organic Reactions in Steroid Chemistry, from Nostrand Reinhold Company 1972, p. 265-374).

The dienol ether bromination of compounds 3, 12 or 17 can for example be carried out as for the specification from Steroids 1, 233 (1963). Hydrogen bromide elimination with formation of the compounds with the general chemical formulae 4, 13 or 18 is achieved by heating the 6-bromo compound with basic reagents, for example with LiBr or Li₂CO₃, in aprotic solvents, such as dimethylformamide, at temperatures of 50-120° C. or alternatively by heating the 6-bromo compounds in a solvent, such as collidine or lutidine.

The introduction of a substituent R⁴ can be carried out, for example, starting from a compound with one of the general chemical formulae 6, 11, 13, 14, 16 or 18, by epoxidation of the 4,5-double bond with hydrogen peroxide under alkaline conditions and reaction of the resultant epoxides in a suitable solvent with acids with the general chemical formula H—R⁴, where R⁴ can be a halogen atom, preferably chlorine or bromine. Compounds in which R⁴ has the meaning bromine can for example be reacted with 2,2-difluoro-2-(fluorosulfonyl)methyl acetate in dimethylformamide in the presence of copper(I) iodide to compounds in which R⁴ has the meaning fluorine. Alternatively, starting from a compound with one of the general chemical formulae 6, 11, 13, 14, 16 or 18, halogen can be introduced directly by reaction with sulfuryl chloride or sulfuryl bromide in the presence of a suitable base, for example pyridine, with R⁴ having the meaning chlorine or bromine.

Compound 4 is converted by methenylation of the 6,7-double bond by known methods, for example with dimethylsulfoxonium methylide (see for example DE-A 11 83 500, DE-A 29 22 500, EP-A 0 019 690, U.S. Pat. No. 4,291,029; J. Am. Chem. Soc. 84, 867 (1962)) to a compound 5 (R⁶, R⁷ together form a methylene group), obtaining a mixture of the α- and β-isomers, which can be separated into the individual isomers for example by chromatography.

Compounds of type 5 can be obtained as described in the examples or similarly to these specifications, using similar reagents to those described there.

Synthesis of the spirocyclic compound 18 (R^{6a}, R^{6b} together form 1,2-ethanediyl) starts from compounds 11 or 14, which are first converted to a 3-amino-3,5-diene derivative 15 (X=NR^{21a}R^{21b}) By reaction with formalin in alcoholic solution, the 6-hydroxymethylene derivative 16 (R⁶=hydroxymethylene) is obtained. After converting the hydroxyl group into a leaving group, such as a mesylate, tosylate or even benzoate, compound can be prepared by reaction with trimethylsulfoxonium iodide using bases, such as alkali hydroxides, alkali alcoholates, in suitable solvents, such as dimethyl sulfoxide.

For introduction of a 6-methylene group, compound 16 (R⁶=hydroxymethylene) can be dehydrated with for example hydrochloric acid in dioxane/water. Compound 18 (R^{6a}, R^{6b} together form methylene) can also be produced after converting the hydroxyl group into a leaving group, such as a mesylate, tosylate or even benzoate (see DE-A 34 02 329, EP-A 0 150 157, U.S. Pat. No. 4,584,288; J. Med. Chem. 34, 2464 (1991)).

Another possibility for the production of 6-methylene compounds 18 is the direct reaction of the 4(5)-unsaturated 3-ketones, for example of compound 16 (R⁶=hydrogen), with formaldehyde acetals in the presence of sodium acetate with for example phosphorus oxychloride or phosphorus pentachloride in suitable solvents, such as chloroform (see for example K. Annen, H. Hofineister, H. Laurent and R. Wiechert, Synthesis 34 (1982)).

The 6-methylene compounds can be used for the preparation of compounds with the general chemical formula 18, in which R^{6a} is methyl and R^{6b} and R⁷ together form an additional bond.

For this it is possible for example to use a method described in Tetrahedron 21, 1619 (1965), in which isomerization of the double bond is achieved by heating the 6-methylene compounds in ethanol with 5% palladium/charcoal catalyst, pretreated either with hydrogen or by heating with a small amount of cyclohexene. The isomerization can also be carried out with a catalyst that has not been pretreated, if a small amount of cyclohexene is added to the reaction mixture. The formation of small proportions of hydrogenated products can be prevented by adding an excess of sodium acetate.

Alternatively, compound 17 (X═OR²²) can be used as precursor. The direct preparation of 6-methyl-4,6-dien-3-one derivatives has been described (see K. Annen, H. Hofineister, H. Laurent and R. Wiechert, Lieb. Ann. 712 (1983)).

Compounds 18 in which R^{6b} represents an α-methyl function can be prepared in suitable conditions from the 6-methylene compounds (18: R^{6a}, R^{6b} together form methylene) by hydrogenation. The best results (selective hydrogenation of the exo-methylene function) are achieved by transfer-hydrogenation (J. Chem. Soc. 3578 (1954)). If the 6-methylene derivatives 18 are heated in a suitable solvent, for example ethanol, in the presence of a hydride donor, for example cyclohexene, then 6α-methyl derivatives are obtained at very good yields. Small proportions of 6β-methyl compound can be isomerized in acid conditions (Tetrahedron 1619 (1965)).

The selective preparation of 6β-methyl compounds is also possible. For this, the 4-en-3-ones, such as compound 16, are reacted for example with ethylene glycol, trimethyl orthoformate in dichloromethane in the presence of catalytic amounts of an acid, for example p-toluenesulfonic acid, to the corresponding 3-ketals. During this ketalization there is isomerisation of the double bond into position C⁵. Selective epoxidation of this 5-double bond is achieved for example by using organic per-acids, for example m-chloroperbenzoic acid, in a suitable solvent, such as dichloromethane. As an alternative, the epoxidation can also be carried out with hydrogen peroxide in the presence of for example of hexachloroacetone or 3-nitrotrifluoroacetophenone. The 5,6α-epoxides formed can then be opened axially using appropriate alkylmagnesium halides or alkyllithium compounds. In this way, 5α-hydroxy-6β-alkyl compounds are obtained. The 3-keto protecting group can be cleaved, obtaining the 5α-hydroxy function, by treatment in mild acidic conditions (acetic acid or 4N hydrochloric acid at 0° C.). Basic elimination of the 5α-hydroxy function with for example dilute aqueous sodium hydroxide solution yields the 3-keto-4-ene compounds with a 6-alkyl group in the β position. Alternatively, cleavage of the ketal in harsher conditions (with aqueous hydrochloric acid or another strong acid) yields the corresponding 6α-alkyl compounds.

The introduction of a 7-alkyl, 7-alkenyl or 7-alkynyl group to form compounds with the general chemical formula 14 is effected by 1,6-addition of a corresponding organometallic compound to the precursor with the general chemical formula 13 under the action of copper salts. Divalent metals, such as magnesium and zinc, are preferred; chlorine, bromine and iodine are preferred as counterions. Suitable copper salts are monovalent or divalent copper compounds, for example copper chloride, copper bromide or copper acetate. The reaction takes place in an inert solvent, for example tetrahydrofuran, diethyl ether or dichloromethane.

The compounds 6, 11, 13, 14, 16, 18 or 20 obtained, in which Z stands for an oxygen atom, can be converted by reaction with hydroxylamine hydrochloride, alkyloxyamine hydrochlorides or sulfonyl hydrazines in the presence of a tertiary amine at temperatures from −20 to +40° C. to their corresponding E/Z-configured oximes or sulfonyl hydrazones (general chemical formula I with Z denoting NOR¹, NNHSO₂R¹)). Suitable tertiary bases are for example trimethylamine, triethylamine, pyridine, N,N-dimethylaminopyridine, 1,5-diazabicyclo[4.3.0]non-5-ene (DBN) and 1,5-diazabicyclo[5.4.0]undec-5-ene (DBU), pyridine being preferred. An analogous method is described for example in WO-A 98/24801 for the production of corresponding 3-oxyimino derivatives of drospirenone.

For the preparation of an end product with the general chemical formula I with Z denoting two hydrogen atoms, the 3-oxo group can be removed for example following the instructions given in DE-A 28 05 490 by reductive cleavage of a thioketal of the 3-keto compound on a suitable precursor, for example of compounds with one of the general chemical formulae 6, 11, 13, 14, 16, 18 or 20.

The formation of spirolactones to compounds with one of the general chemical formulae 6 or 11 is carried out starting from the corresponding 17-hydroxypropenyl compounds 5 or 10, by oxidation. Oxidation processes that may be mentioned are for example the Jones oxidation, oxidation with potassium permanganate for example in an aqueous system of tert.-butanol and sodium dihydrogen phosphate, oxidation with sodium chlorite in aqueous tert.-butanol, optionally in the presence of a chlorine trap, for example in the presence of 2-methyl-2-butene, or by oxidation with manganese dioxide.

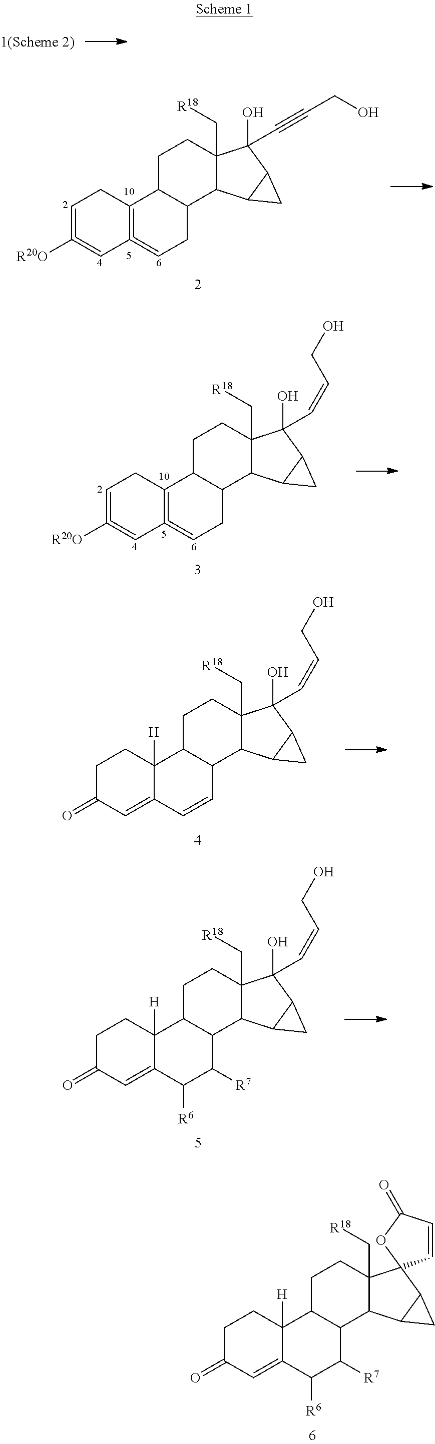

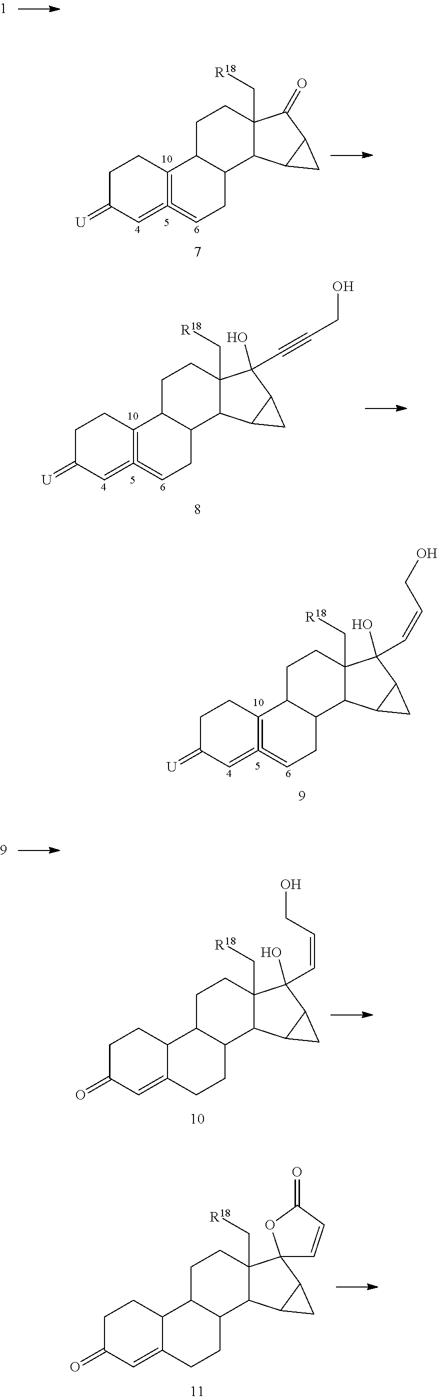

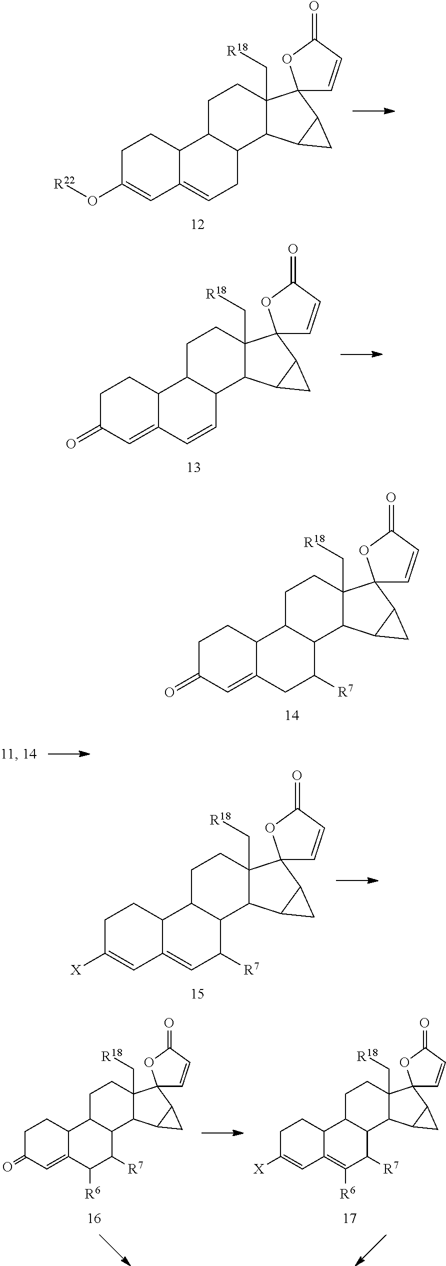

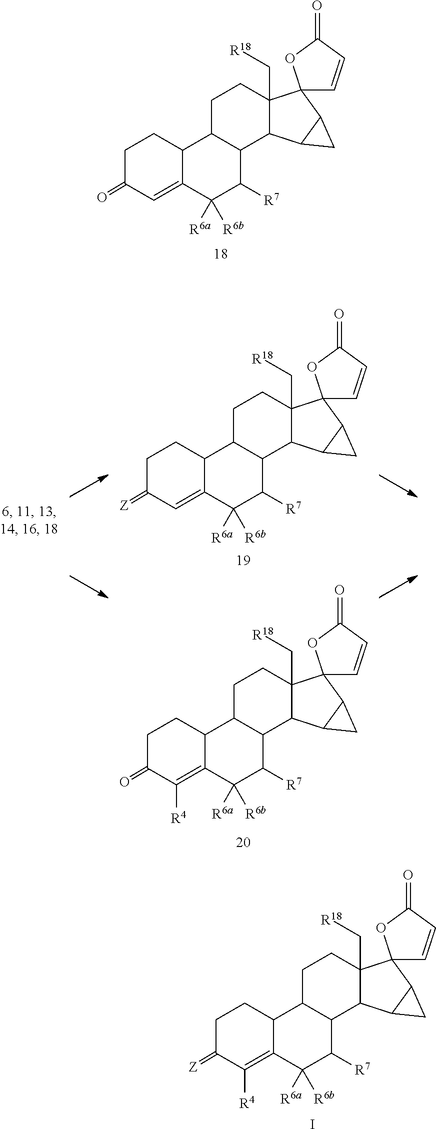

Compound 1 in Scheme 2 has a double bond between C⁵ and C⁶ or between C⁵ and C¹⁰ and another double bond between C² and C³ or between C³ and C⁴.

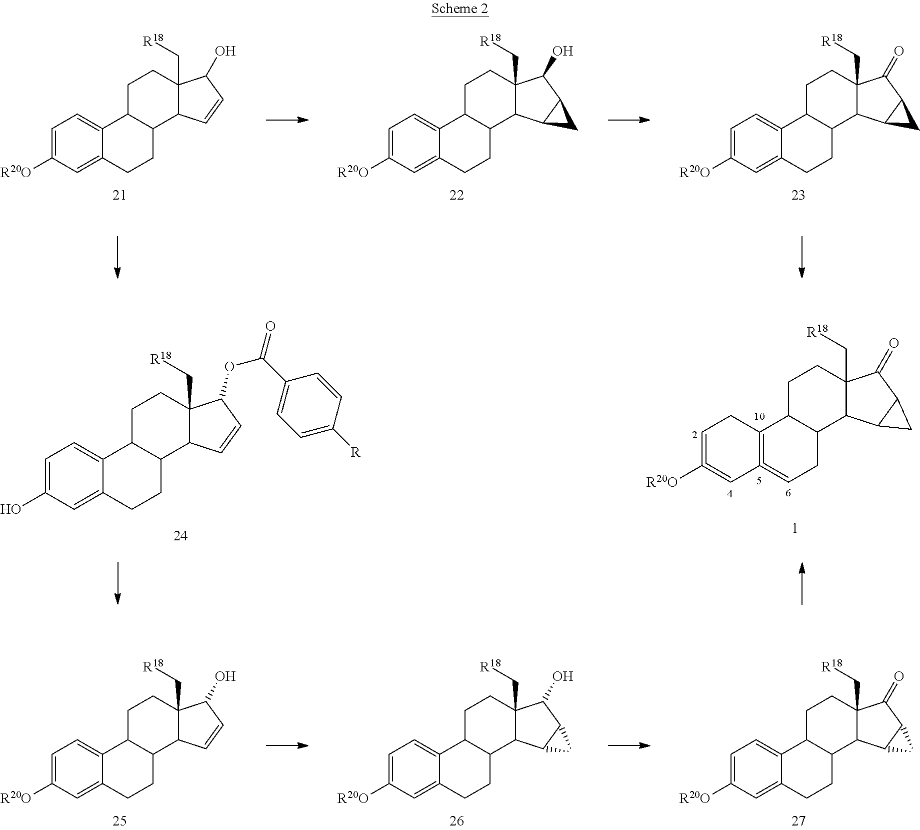

The following examples are provided for further explanation of the invention, without it being limited to the examples shown:

### EXAMPLE 1

### 17-spirolactonization with Manganese Dioxide

### 6β,7β;15β,16β-Bismethylene-17β-hydroxy-18-methyl-19-nor-17α-pregna-4,20(Z)-dien-3-one-21-carboxylic acid γ-lactone

5.87 g manganese dioxide was added to a solution of 865 mg of compound A prepared according to Example 1a in 61 ml dichloromethane and stirred for approx. 16 hours at 23° C. It was filtered on Celite, and after concentration by evaporation and chromatography, 834 mg of the title compound was isolated as a crystalline solid.

¹H-NMR (CDCl₃): δ=0.6-0.71 (2H), 0.88 (3H), 0.92-1.12 (3H), 1.29-1.99 (13H), 2.12-2.52 (4H), 6.09 (1H), 6.17 (1H), 7.51 (1H) ppm.

### EXAMPLE 1a

### Corey Cyclopropanation

### 6β,7β;15β,16β-Bismethylene-17α(Z)-(3′-hydroxypropen-1′-yl)-18-methyl-17β-hydroxyestra-4-en-3-one (A) and 6α,7α; 15β,16β-Bismethylene-17α(Z)-(3′-hydroxypropen-1′-yl)-18-methyl-17β-hydroxyestra-4-en-3-one (B)

3.33 g of a 55% suspension of sodium hydride in white oil was added in portions at 23° C. to a solution of 16.8 g sulfoxonium iodide in 373 ml dimethylsulfoxide. The mixture was stirred for a further 2 hours, 10.6 g of the compound prepared according to Example 1b was added and it was left to react for a further 2.5 hours. The mixture was poured into water, extracted several times with ethyl acetate, the combined organic extracts were washed with saturated sodium chloride solution and dried over sodium sulfate. The residue obtained after filtration and removal of the solvent was purified and separated by chromatography. 1.51 g of the title compound A and 1.01 g of the title compound B were isolated.

### EXAMPLE 1b

### Dienone Formation from Dienol Ether

### 17α(Z)-(3′-Hydroxypropen-1′-yl)-18-methyl-15β,16β-methylene-17β-hydroxyestra-4,6-dien-3-one

1.03 g sodium acetate, 10 ml water and, in portions, a total of 4.23 g dibromohydantoin were added, at 3° C., to a solution of 10.4 g of the compound prepared according to Example 1c in 125 ml dimethylformamide. After 30 minutes, 3.91 g lithium bromide and 3.42 g lithium carbonate were added to the mixture and it was heated for 3 hours at a bath temperature of 100° C. The mixture was poured into water and extracted several times with ethyl acetate. The combined organic extracts were washed with saturated sodium chloride solution and dried over sodium sulfate. The residue obtained after filtration and removal of the solvent was purified by chromatography. 6.35 g of the title compound was isolated.

### EXAMPLE 1c

### Lindlar Hydrogenation

### 17α(Z)-(3′-Hydroxypropen-1′-yl)-3-methoxy-18-methyl-15β,16β-methylene-17β-hydroxyestra-3,5-diene

100 ml pyridine and 5 g palladium on barium sulfate were added to a solution of 75 g of the compound prepared according to Example 1d in 1.5 l tetrahydrofuran and it was hydrogenated in a hydrogen atmosphere. It was filtered on Celite and, after concentration by evaporation, 75.7 g of the title compound was isolated, and was reacted further without purification.

### EXAMPLE 1d

### Hydroxypropyne Addition

### 17α(Z)-(3′-Hydroxypropyn-1′-yl)-3-methoxy-18-methyl-15β,16β-methylene-17β-hydroxyestra-3,5-diene

1 l of a 2.5-molar solution of buthyllithium in hexane was added to a solution of 83 ml of 2-propyn-1-ol in 1 l tetrahydrofuran at −78° C. After 30 minutes a solution of 90 g of 3-methoxy-18-methyl-15β,16β-methylene-estra-3,5-dien-17-one in 0.5 l tetrahydrofuran was added dropwise, allowed to warm up to 23° C. and stirred for a further 3 hours. The mixture was poured into saturated, ice-cold ammonium chloride solution, extracted several times with ethyl acetate, the combined organic extracts were washed with saturated sodium chloride solution and dried over sodium sulfate. The residue obtained after filtration and removal of the solvent was purified by crystallization. 90.3 g of the title compound was isolated.

### EXAMPLE 2

### 6α,7α;15β,16β-Bismethylene-17β-hydroxy-18-methyl-19-nor-17α-pregna-4,20(Z)-dien-3-one-21-carboxylic acid γ-lactone

Similarly to Example 1, 114 mg of compound B prepared according to Example 1a was reacted, and after processing and purification, 68 mg of the title compound was isolated.

¹H-NMR (CDCl₃): δ=0.58 (1H), 0.77-1.04 (5H), 0.85 (3H), 1.25 (1H), 1.39 (1H), 1.49 (1H), 1.54-1.91 (7H), 1.99-2.18 (4H), 2.26 (1H), 2.49 (1H), 1.99 (2H), 7.48 (1H) ppm.

### EXAMPLE 3

### 17β-Hydroxy-18-methyl-15β,16β-methylene-19-nor-17α-pregna-4,6,20(Z)-trien-3-one-21-carboxylic acid γ-lactone

Similarly to Example 1, 5 g of the compound prepared according to Example 1a was, reacted, and after processing and purification, 4.12 g of the title compound was isolated.

¹H-NMR (CDCl₃): δ=0.63 (1H), 0.88 (3H), 0.94 (1H), 1.05-1.34 (3H), 1.45-1.93 (7H), 2.16-2.60 (6H), 5.81 (1H), 6.05 (1H), 6.29 (1H), 6.45 (1H), 7.45 (1H) ppm.

### EXAMPLE 4

### 1,6-Addition

### 7α,18-Bismethyl-17β-hydroxy-15β,16β-methylene-19-nor-17α-pregna-4,20(Z)-dien-3-one-21-carboxylic acid γ-lactone (A) and 7β,18-Bismethyl-17β-hydroxy-15β,16β-methylene-19-nor-17α-pregna-4,20(Z)-dien-3-one-21-carboxylic acid γ-lactone (B)

3.6 ml of a 3-molar solution of methylmagnesium chloride in tetrahydrofuran was added dropwise to a suspension of 87 mg copper(I) chloride in 15 ml tetrahydrofuran cooled to −30° C., and the solution was stirred for a further 10 minutes. The solution was cooled to −25° C. and added dropwise to 1.5 g of the compound prepared according to Example 3 in 70 ml tetrahydrofuran. After 1 minute it was poured into 1N hydrochloric acid, extracted several times with ethyl acetate, the combined organic extracts were washed with saturated sodium chloride solution and dried over sodium sulfate. The residue obtained after filtration and removal of the solvent was purified by chromatography. 20.8 mg of the title compound A and 109 mg of the title compound B were isolated.

¹H-NMR (CDCl₃) of A: δ=0.55 (1H), 0.86 (3H), 0.91 (3H), 0.83-0.94 (1H), 1.04 (1H), 1.14-1.28 (2H), 1.41-1.75 (5H), 1.78-1.95 (3H), 2.03-2.15 (2H), 2.19-2.45 (5H), 2.57 (1H), 5.87 (1H), 6.04 (1H), 7.47 (1H) ppm.

¹H-NMR (CDCl₃) of B: δ=0.62 (1H), 0.86 (3H), 0.90-1.12 (3H), 1.24 (3H), 1.28 (1H), 1.41 (1H), 1.45-1.88 (8H), 2.01-2.30 (5H), 2.37 (1H), 2.49 (1H), 5.83 (1H), 6.02 (1H), 7.44 (1H) ppm.

### EXAMPLE 5

### 17β-Hydroxy-7α-ethyl-18-methyl-15β,16β-methylene-19-nor-17α-pregna-4,20(Z)-dien-3-one-21-carboxylic acid γ-lactone (A) and 17β-Hydroxy-7β-ethyl-18-methyl-15β,16β-methylene-19-nor-17α-pregna-4,20(Z)-dien-3-one-21-carboxylic acid γ-lactone (B)

Similarly to Example 4, 200 mg of the compound prepared according to Example 3 using ethylmagnesium chloride was reacted, and after processing and purification, 59 mg of the title compound A was isolated along with a still contaminated mixture, which contained proportions of the title compound B.

¹H-NMR (CDCl₃) of A: δ=0.63 (1H), 0.86 (3H), 0.96 (3H), 0.89-1.10 (3H), 1.30 (1H), 1.34-1.89 (11H), 2.01-2.42 (6H), 2.62 (1H), 5.85 (1H), 6.02 (1H), 7.43 (1H) ppm.

### EXAMPLE 6

### 17β-Hydroxy-7α-vinyl-18-methyl-15β,16β-methylene-19-nor-17α-pregna-4,20(Z)-dien-3-one-21-carboxylic acid γ-lactone (A) and 17β-Hydroxy-7β-vinyl-18-methyl-15β,16β-methylene-19-nor-17α-pregna-4,20(Z)-dien-3-one-21-carboxylic acid γ-lactone (B)

Similarly to Example 4, 200 mg of the compound prepared according to Example 3 using vinylmagnesium chloride was reacted, and after processing and purification, 41 mg of the title compound A was isolated along with a still contaminated mixture, which contained proportions of the title compound B.

¹H-NMR (CDCl₃) of A: δ=0.61 (1H), 0.90 (3H), 0.94 (1H), 1.10 (1H), 1.24 (1H), 1.33 (1H), 1.46-1.69 (4H), 1.74 (1H), 1.82-2.05 (3H), 2.07-2.38 (4H), 2.48 (1H), 2.56 (1H), 2.70 (1H), 2.87 (1H), 5.22 (1H), 5.27 (1H), 5.92 (2H), 6.07 (1H), 7.49 (1H) ppm.

### EXAMPLE 7

### 17β-Hydroxy-7α-cyclopropyl-18-methyl-15β,16β-methylene-19-nor-17α-pregna-4,20(Z)-dien-3-one-21-carboxylic acid γ-lactone (A) and 17β-Hydroxy-7β-cyclopropyl-18-methyl-15β,16β-methylene-19-nor-17α-pregna-4,20(Z)-dien-3-one-21-carboxylic acid γ-lactone (B)

Similarly to Example 4, 200 mg of the compound prepared according to Example 3 using cyclopropylmagnesium bromide was reacted, and after processing and purification, 49.2 mg of the title compound A was isolated along with a still contaminated mixture, which contained proportions of the title compound B.

¹H-NMR (CDCl₃) of A: δ=0.09 (1H), 0.38 (1H), 0.49-0.68 (4H), 0.87 (3H), 0.93 (1H), 1.08 (1H), 1.23 (1H), 1.4.2-1.63 (6H), 1.71 (1H), 1.83 (1H), 1.88 (1H), 1.99 (1H), 2.13 (1H), 2.24-2.32 (2H), 2.37 (1H), 2.40-2.49 (2H), 2.53 (1H), 5.91 (1H), 6.05 (1H), 7.50 (1H) ppm.

### EXAMPLE 8

### 4-chlorination

### 6β,7β;15β,16β-Bismethylene-4-chloro-17β-hydroxy-18-methyl-19-nor-17α-pregna-4,20(Z)-dien-3-one-21-carboxylic acid γ-lactone

19 μl sulfuryl chloride was added at 3° C. to a solution of 50 mg of the compound prepared according to Example in 0.5 ml pyridine and stirred for a further 1.5 hours at 3° C. The solution was poured into saturated sodium hydrogencarbonate solution, extracted several times with ethyl acetate, the combined organic extracts were washed with saturated sodium chloride solution and dried over sodium sulfate. The residue obtained after filtration and removal of the solvent was purified by chromatography. 38 mg of the title compound was isolated.

¹H-NMR (CDCl₃): δ=0.67 (1H), 0.88 (3H), 0.95-1.21 (3H), 1.35 (1H), 1.46-1.80 (10H), 1.88 (1H), 2.05 (1H), 2.14-2.51 (4H), 2.73 (1H), 6.10 (1H), 7.52 (1H) ppm.

### EXAMPLE 9

### 17β-Hydroxy-18-methyl-15β,16β-methylene-19-nor-17α-pregna-4,20(Z) dien-3-one-21-carboxylic acid γ-lactone

Similarly to Example 1, 200 mg of the compound prepared according to Example 9a was reacted, and after processing and purification, 186 mg of the title compound was isolated.

¹H-NMR (CDCl₃): δ=0.55 (1H), 0.86 (3H), 0.85-0.99 (2H), 1.06 (1H), 1.17-1.33 (2H), 1.40-1.89 (8H), 2.02-2.46 (7H), 2.57 (1H), 5.86 (1H), 6.03 (1H), 7.45 (1H) ppm.

### EXAMPLE 9a

### Enone Formation from Dienol Ether with Oxalic Acid

17α(Z)-(3′-Hydroxypropen-1′-yl)-18-methyl-15β,16β-methylene-17β-hydroxyestra-4-en-3-one

50 ml of a saturated aqueous solution of oxalic acid was added to a suspension of 5.0 g of the compound prepared according to Example 1c in 30 ml acetone and 30 ml water; 30 ml methanol and 50 ml tetrahydrofuran were added, and the suspension was stirred for 0.5 hours at 23° C. The suspension was poured into saturated sodium hydrogencarbonate solution, extracted several times with ethyl acetate, the combined organic extracts were extracted with saturated sodium chloride solution and dried over sodium sulfate. The residue obtained after filtration and removal of the solvent was purified by chromatography. 4.26 g of the title compound was isolated.

### EXAMPLE 10

### Introduction of 6-hydroxymethyl

### 17β-Hydroxy-6β-hydroxymethylene-18-methyl-15β,16β-methylene-19-nor-17α-pregna-4,20(Z)-dien-3-one-21-carboxylic acid γ-lactone

370 μl of a 37% aqueous formaldehyde solution was added to a solution of 382 mg of the compound prepared according to Example 10a in a mixture of 3.5 ml toluene and 7.8 ml ethanol and stirred for 6 hours at 23° C. The solution was concentrated by evaporation, and the residue was purified by chromatography. 119 mg of the title compound was isolated.

¹H-NMR (CDCl₃): δ=0.61 (1H), 0.90 (3H), 0.86-1.18 (3H), 1.29 (1H), 1.45-1.97 (10H), 2.06 (1H), 2.18 (1H), 2.22-237 (3H), 2.47 (1H), 2.77 (1H), 3.80 (2H), 5.99 (1H), 6.08 (1H), 7.49 (1H) ppm.

### EXAMPLE 10a

### Dienamine Formation

### 17β-Hydroxy-18-methyl-15β,16β-methylene-3-pyrrolidinyl-19-nor-17α-pregna-3,5,20(Z)-triene-21-carboxylic acid γ-lactone

271 μl pyrrolidine was added to a solution of 500 mg of the compound prepared according to Example 9 in 5 ml methanol and heated under reflux for 2 hours. The solution was cooled, the precipitate was filtered off with suction, washed with a little cold methanol, and 390 mg of the title compound was obtained, and this was reacted further without additional purification.

### EXAMPLE 11

### 6-spirocyclopropanation

### 6,6-(1,2-Ethanediyl)-17β-hydroxy-18-methyl-15β,16β-methylene-19-nor-17α-pregna-4,20(Z)-dien-3-one-21-carboxylic acid γ-lactone

100 mg trimethylsulfoxonium iodide was dissolved in 2.0 ml dimethylsulfoxide, 18 mg of a 60% sodium hydride dispersion was added to the mixture and it was stirred for 2 hours at 23° C. Then a solution of 58 mg of the compound prepared according to Example 11a in 1.0 ml dimethylsulfoxide was added dropwise and it was stirred for a further 2.5 hours at 23° C. The mixture was poured into water, extracted several times with ethyl acetate, the combined organic extracts were washed with water and saturated sodium chloride solution and dried over sodium sulfate. The residue obtained after filtration and removal of the solvent was purified by chromatography. 25.4 mg of the title compound was isolated.

¹H-NMR (CDCl₃): δ=0.44 (1H), 0.53 (1H), 0.62 (1H), 0.80 (1H), 0.88 (3H), 0.90-1.03 (2H), 1.10 (1H), 1.17-1.89 (11H), 1.92-2.30 (5H), 2.42 (1H), 5.72 (1H), 6.04 (1H), 7.45 (1H) ppm.

### EXAMPLE 11a

### 6-tosyloxymethyl Formation

### 17β-Hydroxy-18-methyl-15β,16β-methylene-6β-(p-tolylsulfonyloxymethyl)-19-nor-17α-pregna-4,20(Z)-dien-3-one-21-carboxylic acid γ-lactone

0.65 ml triethylamine and 175 mg p-toluenesulfonic acid chloride were added to a solution of 150 mg of the compound prepared according to Example 10 in 7 ml dichloromethane and stirred for 6 hours at 23° C. The solution was poured into saturated sodium carbonate solution, extracted several times with ethyl acetate, the combined organic extracts were washed with water and saturated sodium chloride solution and dried over sodium sulfate. The residue obtained after filtration and removal of the solvent was purified by chromatography. 134 mg of the title compound was isolated.

### EXAMPLE 12

### 17β-Hydroxy-15α,16α-methylene-19-nor-17α-pregna-4,20(Z)-dien-3-one-21-carboxylic acid γ-lactone

Similarly to Example 1, 750 mg of the compound prepared according to Example 12a was reacted, and after processing and purification, 484 mg of the title compound was isolated.

¹H-NMR (CDCl₃): δ=0.76-0.89 (3H), 1.03 (1H), 1.14 (1H), 1.21-1.33 (2H), 1.37 (3H), 1.39 (1H), 1.45-1.62 (2H), 1.69 (1H), 1.75-1.91 (2H), 2.11-2.49 (6H), 2.58 (1H), 5.89 (1H), 6.06 (1H), 7.40 (1H) ppm.

### EXAMPLE 12a

### 17α(Z)-(3′-Hydroxypropen-1′-yl)-15α,16α-methylene-17β-hydroxyestra-4-en-3-one

0.83 ml of 4N hydrochloric acid was added to a solution of 300 mg of the compounds prepared according to Example 12b in 15 ml acetone and stirred for 1 hour at 23° C. The solution was poured into saturated sodium hydrogencarbonate solution, extracted several times with ethyl acetate, the combined organic extracts were washed with saturated sodium chloride solution and dried over sodium sulfate. The residue obtained after filtration and removal of the solvent was purified by chromatography. 135 mg of the title compound was isolated.

### EXAMPLE 12b

### 17α(Z)-(3′-Hydroxypropen-1′-yl)-15α,16α-methylene-17β-hydroxyestra-5-en-3-one-3-ethylene ketal and 17α(Z)-(3′-hydroxypropen-1′-yl)-15α,16α-methylene-17β-hydroxyestra-5(10)-en-3-one-3-ethylene ketal

Similarly to Example 1c, 300 mg of the compounds prepared according to Example 12c was reacted, and after processing, 300 mg of the title compounds was isolated, and reacted further without purification.

### EXAMPLE 12c

### 17α(Z)-(3′-Hydroxypropyn-1′-yl)-15α,16α-methylene-17β-hydroxyestra-5-en-3-one-3-ethylene ketal and 17α(Z)-(3′-hydroxypropyn-1′-yl)-15α,16α-methylene-17β-hydroxyestra-5(10)-en-3-one-3-ethylene ketal

Similarly to Example 1d, 278 mg of the compounds prepared according to Example 12d was reacted and after processing, 347 mg of the title compounds was isolated, and reacted further without purification.

### EXAMPLE 12d

### 15α,16α-Methylene-estra-5-ene-3,17-dione-3-ethylene ketal and 15α,16α-methylene-estra-5(10)-ene-3,17-dione-3-ethylene ketal

A spatula tip of molecular sieve 4 Å, 700 mg of N-methylmorpholino-N-oxide and 90 mg tetrabutylammonium perruthenate were added to a solution of 1.06 g of the compounds prepared according to Example 12e in 32 ml dichloromethane and stirred at 23° C. for approx. 16 hours. The mixture was concentrated by evaporation, and the residue was purified by chromatography. 878 mg of the title compounds was isolated.

### EXAMPLE 12e

### Conversion of 3-enol Ether to Ethylene Ketal

15α,16α-Methylene-17α-hydroxyestra-5-en-3-one-3-ethylene ketal and 15α,16α-methylene-17α-hydroxyestra-5(10)-en-3-one-3-ethylene ketal

10 ml ethylene glycol and 4.4 mg p-toluenesulfonic acid hydrate were added to a solution of 500 mg of the compound prepared according to Example 12f in 10 ml tetrahydrofuran and stirred at 23° C. for 2 hours. The solution was poured into saturated sodium hydrogencarbonate solution, extracted several times with ethyl acetate, the combined organic extracts were washed with saturated sodium chloride solution and dried over sodium sulfate. The residue obtained after filtration and removal of the solvent was purified by chromatography. 359 mg of the title compound was isolated.

### EXAMPLE 12f

### Birch Reduction

### 3-Methoxy-15α,16α-methylene-17α-hydroxyestra-2,5(10)-diene

9.91 g lithium was added to 597 ml ammonia at −75° C., and within 1 hour a solution of 24.6 g of the compound prepared according to Example 12g in 1.2 l tetrahydrofuran was added dropwise. 720 ml ethanol was added to the mixture, after 1 hour it was allowed to warm up to −50° C. and was stirred for a further 2 hours. Then 600 ml water was added, it was allowed to warm up to 23° C., it was extracted several times with ethyl acetate, the combined organic extracts were washed with saturated sodium chloride solution and dried over sodium sulfate. After filtration and removal of the solvent, 27.1 g of the title compound was isolated, and was reacted further without purification.

### EXAMPLE 12g

### 3-Methoxy-15α,16α-methylene-17α-hydroxyestra-1,3,5(10)-triene

86.6 g zinc dust was added to a suspension of 1.5 g copper(II) acetate in 900 ml diethyl ether and heated under reflux for 10 minutes. Then 11.7 ml diiodomethane was added and it was heated under reflux for a further minutes. A solution of 37.6 g of the compound prepared according to Example 12h was put in 100 ml tetrahydrofuran, and, spread over a total of 40 hours, a further 35 ml diiodomethane in total was added. The cooled mixture was filtered on Celite, the filtrate was Washed with saturated sodium chloride solution and dried over sodium sulfate. The residue obtained after filtration and removal of the solvent was purified by recrystallization. 24.6 g of the title compound was isolated.

### EXAMPLE 12h

### Benzoate Saponification

### 3-Methoxy-17α-hydroxyestra-1,3,5(10),15-tetraene

75.5 g potassium carbonate was added to a solution of 96.3 g of the compound prepared according to Example 12i in 1.1 l methanol, and was stirred at 50° C. for 2 hours. The solution was concentrated by evaporation, water was added, it was extracted several times with ethyl acetate, the combined organic extracts were washed with water and dried over sodium sulfate. The residue obtained after filtration and removal of the solvent was purified by recrystallization. 46 g of the title compound was isolated.

### EXAMPLE 12i

### Mitsunobu

### 4-Nitrobenzoic acid 3-methoxy-estra-1,3,5(10)15-tetraen-17-yl ester

121 g triphenylphosphine, 27.1 g 4-nitrobenzoic acid and 30.9 ml diisopropyl azodicarboxylate were added to a solution of 43.9 g of 3-methoxy-17β-hydroxyestra-1,3,5(10),15-tetraene in 1.6 l tetrahydrofuran and stirred at 23° C. for 2 hours. Saturated sodium chloride solution was added to the solution, it was extracted with ethylacetate, the combined organic extracts were washed with saturated sodium chloride solution and dried over sodium sulfate. The residue obtained after filtration and removal of the solvent was taken up in 1.2 l acetone, 80 ml of 30% hydrogen peroxide solution was added while cooling, and after 20 minutes it was poured, while cooling, into 600 ml of semiconcentrated sodium thiosulfate solution. The mixture was extracted with ethyl acetate, the combined organic extracts were washed with saturated sodium chloride solution and dried over sodium sulfate. The residue obtained after filtration and removal of the solvent was purified by recrystallization. 52.5 g of the title compound was isolated.

### EXAMPLE 13

### 17β-Hydroxy-15α,16α-methylene-19-nor-17α-pregna-4,6,20(Z)-trien-3-one-21-carboxylic acid γ-lactone

Similarly to Example 1b, 1.75 g of the compound prepared according to Example 13a was reacted, and after processing and purification, 1.52 g of the title compound was isolated.

¹H-NMR (CDCl₃): δ=0.82 (1H), 0.92-1.09 (3H), 1.16 (1H), 1.26-1.42 (2H), 1.35 (3H), 1.43-1.63 (2H), 1.72 (1H), 1.89 (1H), 2.19-2.59 (5H), 5.80 (1H), 6.03 (1H), 6.26 (1H), 6.40 (1H), 7.36 (1H) ppm.

### EXAMPLE 13a

### Dienol Ether Formation

### 17β-Hydroxy-3-methoxy-15α,16α-methylene-19-nor-17α-pregna-3,5,20(Z)-triene-21-carboxylic acid γ-lactone

205 mg pyridinium p-toluenesulfonate was added to a solution of 1.84 g of the compound prepared according to Example 12 in 20 ml 2,2-dimethoxypropane and was heated under reflux 4 for hours. The solution was poured into saturated sodium hydrogencarbonate solution, extracted several times with ethyl acetate, the combined organic extracts were extracted with saturated sodium chloride solution and dried over sodium sulfate. The residue obtained after filtration and removal of the solvent was purified by recrystallization. 1.75 g of the title compound was isolated.

### EXAMPLE 14

### 17β-Hydroxy-7α-methyl-15α,16α-methylene-19-nor-17α-pregna-4,20(Z)-dien-3-one-21-carboxylic acid γ-lactone (A) and 17β-Hydroxy-7β-methyl-15α,16α-methylene-19-nor-17α-pregna-4,20(Z)-dien-3-one-21-carboxylic acid γ-lactone (B)

Similarly to Example 4, 250 mg of the compound prepared according to Example 13 was reacted and after processing and purification, 97 mg of the title compound A was isolated along with a still contaminated mixture, which contained proportions of the title compound B.

¹H-NMR (CDCl₃) of A: δ=0.74 (1H), 0.85 (3H), 0.88-1.36 (6H), 1.33 (3H), 1.41-1.59 (2H), 1.75-1.92 (3H), 2.05 (1H), 2.19-2.45 (5H), 2.56 (1H), 5.85 (1H), 6.02 (1H), 7.39 (1H) ppm.

### EXAMPLE 15

### 17β-Hydroxy-7α-ethyl-15α,16α-methylene-19-nor-17α-pregna-4,20(Z)-dien-3-one-21-carboxylic acid γ-lactone (A) and 17β-Hydroxy-17β-ethyl-15α,16α-methylene-19-nor-17α-pregna-4,20(Z)-dien-3-one-21-carboxylic acid γ-lactone (B)

Similarly to Example 4, 250 mg of the compound prepared according to Example 13 using ethylmagnesium chloride was reacted, and after processing and purification, 105 mg of the title compound A was isolated along with a still contaminated mixture, which contained proportions of the title compound B.

¹H-NMR (CDCl₃) of A: δ=0.78 (1H), 0.93-1.61 (10H), 0.97 (3H), 1.38 (3H), 1.79-2.04 (4H), 2.11 (1H), 2.24-2.49 (4H), 2.66 (1H), 5.91 (1H), 6.06 (1H), 7.43 (1H) ppm.

### EXAMPLE 16

### 17β-Hydroxy-7α-vinyl-15α,16α-methylene-19-nor-17α-pregna-4;20(Z)-dien-3-one-21-carboxylic acid γ-lactone (A) and 17β-Hydroxy-7β-vinyl-15α,16α-methylene-19-nor-17α-pregna-4,20(Z)-dien-3-one-21-carboxylic acid γ-lactone (B)

Similarly to Example 4, 250 mg of the compound prepared according to Example 13 using vinylmagnesium chloride was reacted, and after processing and purification, 37 mg of the title compound A and 6 mg of the title compound B were isolated.

¹H-NMR (CDCl₃) of A: δ=0.66 (1H), 0.86 (1H), 0.93 (1H), 1.02 (1H), 1.09-1.34 (3H), 1.33 (3H), 1.45-1.57 (2H), 1.75-1.88 (2H), 1.91 (1H), 2.12 (1H), 2.22-2.33 (2H), 2.42 (1H), 2.51 (1H), 2.61 (1H), 2.81 (1H), 5.13 (1H), 5.21 (1H), 5.71 (1H), 5.88 (1H), 6.00 (1H), 7.36 (1H) ppm.

¹H-NMR (CDCl₃) of B: δ=0.66 (1H), 0.76-0.88 (2H), 0.90-1.03 (2H), 1.17-1.36 (2H), 1.31 (3H), 1.48 (1H), 1.57-1.86 (4H), 2.03-2.44 (7H), 4.98 (1H), 5.10 (1H), 5.84 (1H), 5.88 (1H), 6.01 (1H), 7.35 (1H) ppm.

### EXAMPLE 17

### 17β-Hydroxy-7α-cyclopropyl-15α,16α-methylene-19-nor-17α-pregna-4,20(Z)-dien-3-one-21-carboxylic acid γ-lactone (A) and 17β-Hydroxy-7β-cyclopropyl-15α,16α-methylene-19-nor-17α-pregna-4,20(Z)-dien-3-one-21-carboxylic acid γ-lactone (B)

Similarly to Example 4, 250 mg of the compound prepared according to Example 13 using cyclopropylmagnesium bromide was reacted, and after processing and purification, 57 mg of the title compound A and 3 mg of the title compound B were isolated.

¹H-NMR (CDCl₃) of A: δ=−0.03 (1H), 0.43-0.57 (4H), 0.78 (1H), 0.99 (1H), 1.13-1.40 (7H), 1.34 (3H), 1.50 (1H), 1.80-1.96 (3H), 2.12 (1H), 2.21-2.34 (2H), 2.36-2.51 (2H), 2.56 (1H), 5.90 (1H), 6.02 (1H), 7.42 (1H) ppm.

¹H-NMR (CDCl₃) of B: δ=0.06 (1H), 0.44 (1H), 0.49-1.00 (5H), 1.13-1.48 (7H), 1.35 (3H), 1.65-1.98 (4H), 2.09-2.45 (5H), 2.61 (1H), 5.81 (1H), 6.02 (1H), 7.37 (1H) ppm.

### EXAMPLE 18

### 4-Chloro-17β-hydroxy-15α,16α-methylene-19-nor-17α-pregna-4,20(Z)-dien-3-one-21-carboxylic acid γ-lactone

Similarly to Example 8, 80 mg of the compound prepared according to Example 12 was reacted, and after processing and purification, 28 mg of the title compound was isolated.

¹H-NMR (CDCl₃): δ=0.72-0.91 (3H), 0.99 (1H), 1.11 (1H), 1.17-1.40 (3H), 1.33 (3H), 1.48 (1H), 1.52-1.79 (3H), 1.83 (1H), 2.01-2.47 (5H), 2.63 (1H), 3.41 (1H), 6.02 (1H), 7.35 (1H) ppm.

### EXAMPLE 19

### 17β-Hydroxy-15β,16β-methylene-19-nor-17α-pregna-4,20(Z)-dien-3-one-21-carboxylic acid γ-lactone

Similarly to Example 1, 2.9 g of the compound prepared according to Example 19a was reacted, and after processing and purification, 1.73 g of the title compound was isolated.

¹H-NMR (CDCl₃): δ=0.54 (1H), 0.88 (1H), 1.04-1.68 (9H), 1.12 (3H), 1.79 (1H), 1.90 (1H), 2.05-2.46 (6H), 2.56 (1H), 5.86 (1H), 6.02 (1H), 7.43 (1H) ppm.

### EXAMPLE 19a

### 17α(Z)-(3′-Hydroxypropen-1′-yl)-15β,16β-methylene-17β-hydroxyestra-4-en-3-one

Similarly to Example 9a, 14.2 g of the compound prepared according to Example 19b was reacted, and after processing and purification, 11.9 g of the title compound was isolated.

### EXAMPLE 19b

### 17α(Z)-(3′-Hydroxypropen-1′-yl)-3-methoxy-15β,16β-methylene-17β-hydroxyestra-3,5-diene

Similarly to Example 1c, 23.8 g of the compound prepared according to Example 19c was reacted, and after processing and purification, 23.7 g of the title compound was isolated.

### EXAMPLE 19c

### 17α(Z)-(3′-Hydroxypropyn-1′-yl)-3-methoxy-15β,16β-methylene-17β-hydroxyestra-3,5-diene

Similarly to Example 1d, 38 g of 3-methoxy-15β,16β-methylene-estra-3,5-dien-17-one was reacted, and after processing and purification, 39.2 g of the title compound was isolated.

### EXAMPLE 20

### 17β-Hydroxy-15β,16β-methylene-19-nor-17α-pregna-4,6,20(Z)-trien-3-one-21-carboxylic acid γ-lactone

Similarly to Example 1b, 6.9 g of the compound prepared according to Example 20a was reacted, and after processing and purification, 3.2 g of the title compound was isolated.

¹H-NMR (CDCl₃): δ=0.66 (1H), 1.11-1.75 (8H), 1,19 (3H), 1.83 (1H), 2.10 (1H), 2.23-2.48 (4H), 2.59 (1H), 5.86 (1H), 6.09 (1H), 6.33 (1H), 6.48 (1H), 7.47 (1H) ppm.

### EXAMPLE 20a

### 17β-Hydroxy-3-methoxy-15β,16β-methylene-19-nor-17α-pregna-3,5,20(Z)-triene-21-carboxylic acid γ-lactone

Similarly to Example 13a, 6.5 g of the compound prepared according to Example 19 was reacted and 6.9 g of the title compound was isolated, and was reacted further without purification.

### EXAMPLE 21

### 17β-Hydroxy-6β-hydroxymethylene-15β,16β-methylene-19-nor-17α-pregna-4,20(Z)-dien-3-one-21-carboxylic acid γ-lactone

Similarly to Example 10, 727 mg of the compound prepared according to Example 21a was reacted, and after processing and purification, 266 mg of the title compound was isolated.

¹H-NMR (CDCl₃): δ=0.60 (1H), 0.93 (1H), 1.09-1.95 (12H), 1.16 (3H), 2.13-2.53 (5H), 2.77 (1H), 3.80 (2H), 5.99 (1H), 6.07 (1H), 7.47 (1H) ppm.

### EXAMPLE 21a

### 17β-Hydroxy-15β,16β-methylene-3-pyrrolidinyl-19-nor-17α-pregna-3,5,20(Z)-triene-21-carboxylic acid γ-lactone

Similarly to Example 10a, 783 mg of the compound prepared according to Example 19 was reacted, and after processing and purification, 734 mg of the title compound was isolated.

### EXAMPLE 22

### 6,6-(1,2-Ethanediyl)-17β-hydroxy-15β,16β-methylene-19-nor-17α-pregna-4,20(Z)dien-3-one-21-carboxylic acid γ-lactone

Similarly to Example 11, 136 mg of the compound prepared according to Example 22a was reacted, and after processing and purification, 40 mg of the title compound was isolated.

¹H-NMR (CDCl₃): δ=0.44 (1H), 0.52 (1H), 0.61 (1H), 0.80 (1H), 0.98 (1H), 1.11-1.52 (9H), 1.14 (3H), 1.71-1.93 (4H), 2.15-2.28 (3H), 2.41 (1H), 5.71 (1H), 6.02 (1H), 7.43 (1H) ppm.

### EXAMPLE 22a

### 17β-Hydroxy-15β,16β-methylene-6β-(p-tolylsulfonyloxymethyl)-19-nor-17α-pregna-4,20(Z)-dien-3-one-21-carboxylic acid γ-lactone

Similarly to Example 11a, 730 mg of the compound prepared according to Example 0.21 was reacted, and after processing and purification, 143 mg of the title compound was isolated.

### EXAMPLE 23

### 17β-Hydroxy-7α-methyl-15β,16β-methylene-19-nor-17α-pregna-4,20(2)-dien-3-one-21-carboxylic acid γ-lactone (A) and 17β-Hydroxy-7β-methyl-15β, 16β-methylene-19-nor-17α-pregna-4,20(Z)-dien-3-one-21-carboxylic acid γ-lactone (B)

Similarly to Example 4, 250 mg of the compound prepared according to Example 20 was reacted, and after processing and purification, 76 mg of the title compound A was isolated along with a still contaminated mixture, which contained proportions of the title compound B.

¹H-NMR (CDCl₃) of A: δ=0.55 (1H), 0.91 (3H), 1.02-1.58 (8H), 1.13 (3H), 1.73-1.87 (2H), 1.97 (1H), 2.06 (1H), 2.20-2.45 (5H), 2.57 (1H), 5.87 (1H), 6.03 (1H), 7.45 (1H) ppm.

### EXAMPLE 24

### 7α-Ethyl-17β-hydroxy-15β,16β-methylene-19-nor-17α-pregna-4,20(Z)-dien-3-one-21-carboxylic acid γ-lactone (A) and 7β-Ethyl-17β-hydroxy-15β,16β-methylene-19-nor-17α-pregna-4,20(2)-dien-3-one-21-carboxylic acid γ-lactone (B)

Similarly to Example 4, 250 mg of the compound prepared according to Example 20 using ethylmagnesium chloride was reacted, and after processing and purification, 57 mg of the title compound A and 23 mg of the title compound B were isolated.

¹H-NMR (CDCl₃) of A: δ=0.56 (1H), 0.95 (3H), 1.02-1.55 (10H), 1.12 (3H), 1.76-1.86 (2H), 1.93 (1H), 2.02-2.10 (2H), 2.20-2.44 (4H), 2.62 (1H), 5.87 (1H), 6.02 (1H), 7.44 (1H) ppm.

¹H-NMR (CDCl₃) of B: δ=0.63 (1H), 0.96 (3H), 1.00 (1H), 1.06-1.68 (11H), 1.14 (3H), 1.84 (1H), 1.99-2.30 (5H), 2.37 (1H), 260(1H), 5.84 (1H), 6.01 (1H), 7.41 (1H) ppm.

### EXAMPLE 25

### 17β-Hydroxy-15β,16β-methylene-7α-vinyl-19-nor-17α-pregna-4,20(Z)-dien-3-one-21-carboxylic acid γ-lactone (A) and 17β-Hydroxy-15β,16β-methylene-7β-vinyl-19-nor-17α-pregna-4,20(Z)-dien-3-one-21-carboxylic acid γ-lactone (B)

Similarly to Example 4, 250 mg of the compound prepared according to Example 20 using vinylmagnesium chloride was reacted, and after processing and purification, 29 mg of the title compound A was isolated along with a still contaminated mixture, which contained proportions of the title compound B.

¹H-NMR (CDCl₃) of A: δ=0.56 (1H), 1.07 (1H), 1.12 (3H), 1.18-1.31 (4H), 1.39 (1H), 1.49-1.60 (2H), 1.79-1.87 (2H), 1.92 (1H), 2.12 (1H), 2.21-2.33 (2H), 2.43 (1H), 2.51 (1H), 2.66 (1H), 2.83 (1H), 5.17 (1H), 5.22 (1H), 5.85 (1H), 5.87 (1H), 6.01 (1H), 7.43 (1H) ppm.

### EXAMPLE 26

### 7α-Cyclopropyl-17β-hydroxy-15β,16β-methylene-19-nor-17α-pregna-4,20(Z)-dien-3-one-21-carboxylic acid γ-lactone (A) and 7β-Cyclopropyl-17β-hydroxy-15β,16β-methylene-19-nor-17α-pregna-4,20(Z)-dien-3-one-21-carboxylic acid γ-lactone (B)

Similarly to Example 4, 250 mg of the compound prepared according to Example 20 was reacted, and after processing and purification, 91 mg of the title compound A and 25 mg of the title compound B were isolated.

¹H-NMR (CDCl₃) of A: δ=0.07 (1H), 0.39 (1H), 0.47-0.68 (4H), 1.12 (4H), 1.21-1.32 (3H), 1.35-1.61 (5H), 1.83-1.92 (2H), 2.11 (1H), 2.20-2.33 (3H), 2.39-2.49 (2H), 2.53 (1H), 5.90 (1H), 6.03 (1H), 7.48 (1H) ppm.

¹H-NMR (CDCl₃) of B: δ=0.28 (1H), 0.35 (1H), 0.53-0.67 (3H), 0.78-1.12 (4H), 1.14 (3H), 1.21-1.51 (5H), 1.59-1.67 (2H), 1.83 (1H), 1.99-2.30 (5H), 2.39 (1H), 2.57 (1H), 5.82 (1H), 6.02 (1H), 7.43 (1H) ppm.

### EXAMPLE 27

### 4-Chloro-17β-hydroxy-15β,16β-methylene-19-nor-17α-pregna-4,20(Z)-dien-3-one-21-carboxylic acid γ-lactone

Similarly to Example 8, 100 mg of the compound prepared according to Example 19 was reacted, and after processing and purification, 70 mg of the title compound was isolated.

¹H-NMR (CDCl₃): δ=0.61 (1H), 1.01 (1H), 1.11-1.73 (9H), 1.17 (3H), 1.83 (1H), 1.94 (1H), 2.17-2.50 (5H), 2.69 (1H), 3.48 (1H), 6.07 (1H), 7.48 (1H) ppm.

### EXAMPLE 28

### 6,6-(1,2-Ethanediyl)-17β-hydroxy-15α,16α-methylene-19-nor-17α-pregna-4,20(Z)-dien-3-one-21-carboxylic acid γ-lactone

Similarly to Example 11, 115 mg of the compound prepared according to Example 28a was reacted, and after processing and purification, 25 mg of the title compound was isolated.

¹H-NMR (CDCl₃): δ=0.44 (1H), 0.60 (1H), 0.69-1.11 (6H), 1.20-1.53 (6H), 1.35 (3H), 1.65-1.97 (4H), 2.15-2.31 (3H), 2.41 (1H), 5.70 (1H), 6.02 (1H), 7.36 (1H) ppm.

### EXAMPLE 28a

### 17β-Hydroxy-15α,16α-methylene-6β-(p-tolylsulfonyloxymethyl)-19-nor-17α-pregna-4,20(Z)-dien-3-one-21-carboxylic acid γ-lactone

Similarly to Example 11a, 890 mg of the compound prepared according to Example 28b was reacted, and after processing and purification, 115 mg of the title compound was isolated.

### EXAMPLE 28b

### 17β-Hydroxy-6-hydroxymethylene-15α,16α-methylene-19-nor-17α-pregna-4,20(Z)-dien-3-one-21-carboxylic acid γ-lactone

Similarly to Example 10, 895 mg of the compound prepared according to Example 28c was reacted, and after processing, 1.1 g of the title compound was isolated as raw product, and was reacted further without, purification.

### EXAMPLE 28c

### 17β-Hydroxy-15α,16α-methylene-3-pyrrolidinyl-19-nor-17α-pregna-3,5,20(Z)-triene-21-carboxylic acid γ-lactone

Similarly to Example 10a, 858 mg of the compound prepared according to Example 12 was reacted, and after processing and purification, 895 mg of the title compound was isolated.

### EXAMPLE 29

### 6β,7β;15β,16β-Bismethylene-17β-hydroxy-19-nor-17α-pregna-4,20(Z)-dien-3-one-21-carboxylic acid γ-lactone (A) and 6α,7α;15β,16β-Bismethylene-17β-hydroxy-19-nor-17α-pregna-4,20(Z)-dien-3-one-21-carboxylic acid γ-lactone (B)

Similarly to Example 1a, 1.0 g of the compound prepared according to Example 20 was reacted, and after processing and purification, 143 mg of the title compound A and 182 mg of the title compound B were isolated.

¹H-NMR (CDCl₃) of A: δ=0.57-0.63 (2H), 0.98 (1H), 1.04-1.17 (2H), 1.09 (3H), 1.26-1.35 (3H), 1.42-1.77 (7H), 1.88 (1H), 2.10-2.21 (2H), 2.27 (1H), 2.42 (1H), 6.04 (1H), 6.12 (1H), 7.44 (1H) ppm.

¹H-NMR (CDCl₃) of B: δ=0.58 (1H), 0.76-0.86 (2H), 1.00 (1H), 1.04-1.21 (2H), 1.15 (3H), 1.25-1.45 (4H), 1.62-1.69 (2H), 1.76 (1H), 1.87 (1H), 1.98-2.14 (4H), 2.26 (1H), 2.50 (1H), 6.03 (1H), 6.05 (H), 7.46 (1H) ppm.

### EXAMPLE 30

Inert depot systems amenable to intrauterine implantation and composed of a biodegradable polymer or a synthetic silicone polymer consisting of an active ingredient-containing core in the appropriate polymer-active ingredient mixing ratio, surrounded by a polymer membrane ensuring the desired daily release rate, are introduced into the lumen of the rat uterus. The female animals are castrated beforehand and pretreated with estradiol for three days. The implants of different length (5-20 mm) and, a restricted diameter (1.1 to 2 mm) remain for between 4 and 14 days in the rat uterus in order to investigate the local and systemic progestational effect of the released active ingredient on the basis of various parameters in different tissues. The following parameters are measured: 1) local progestational effect on the uterus on the basis of the weight of the uterus, the histologically detectable epithelial height and the expression of progestogen-regulated marker genes (e.g. IGFBP-1); 2) systemic progestational effect on the mammary gland on the basis of the expression of progestogen-regulated marker genes (e.g. RankL), 3) systemic progestational effect on the pituitary on the basis of the LH level (reduction in the estrogen-induced elevation of the LH level).

The compounds of the present invention show a significant progestational effect in the uterus which is comparable to a corresponding treatment with a levonorgestrel-containing depot system such as MIRENA®.

TABLE 1
Receptor binding values

| | | Receptor binding | | | | | |
|---|---|---|---|---|---|---|---|
| | | Progesterone | | Mineralocorticoid | | | |
| | | receptor (PR) | | receptor (MR) | Androgen receptor | | CF |
| | | IC50 | Competition | Competition | IC50 | Competition | PR/CF |
| Ex. | Structure | [nM] | factor | factor | [nM] | factor | MR |
| A | 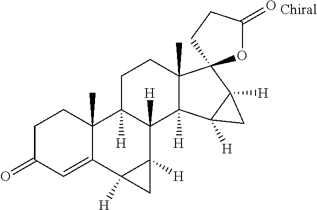 | 43.3 | 2.7 | 0.5 | 630 | 37 | 5.40 |
| 1 | 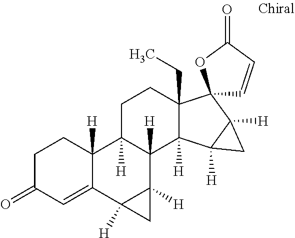 | 41 | 0.76 | 1.6 | 280 | 30.6 | 0.48 |
| 2 | 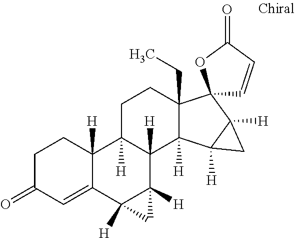 | 630 | 24.37 | 4.4 | 450 | 49.6 | 5.54 |
| 3 | 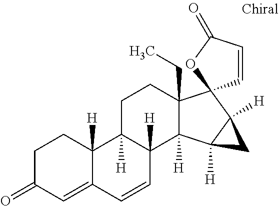 | 72 | 2.72 | 1.5 | 1300 | 62.9 | 1.81 |
| 4A | 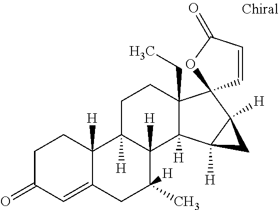 | 48 | 2.66 | 0.6 | 60 | 2.6 | 4.43 |
| 5A | 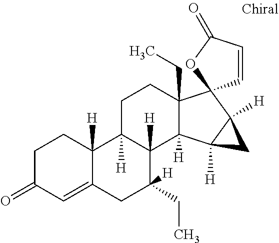 | 300 | 12.00 | 1.0 | 110 | 4.1 | 12.00 |
| 6A | 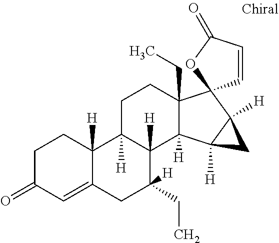 | 120 | 4.85 | 1.5 | 45 | 1.7 | 3.23 |
| 7A | 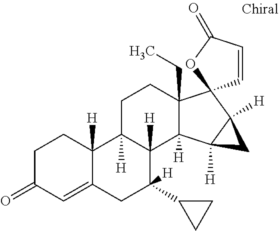 | 380 | 14.22 | 4.7 | 170 | 5.2 | 3.03 |
| 7B | 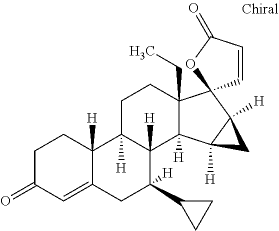 | | | | | | |
| 8 | 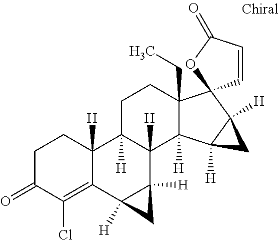 | 740 | 31.12 | 29.0 | 4300 | 240.0 | 1.07 |
| 9 | 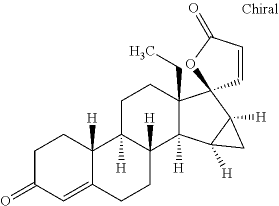 | 78.5 | 1.65 | 1.1 | 0 | 5.6 | 1.50 |
| 10 | 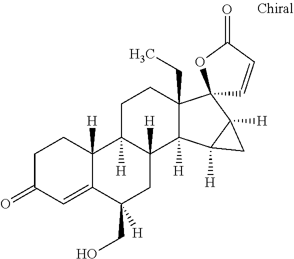 | 167 | 4000.00 | 2.7 | 1000 | 1000.0 | inactive |
| 12 | 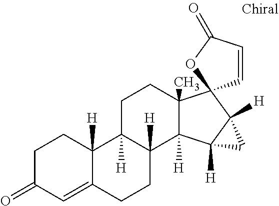 | 49 | 1.50 | 2.9 | 200 | 16.5 | 0.52 |
| 13 | 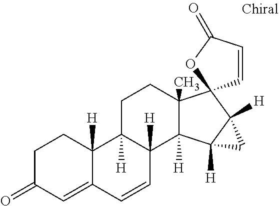 | 190 | 7.89 | 1.6 | 5100 | 163.2 | 4.93 |
| 14A | 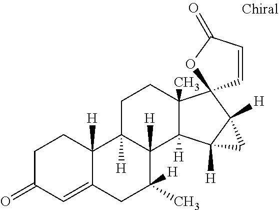 | 49 | 2.04 | 1.3 | 74 | 2.3 | 1.57 |
| 15A | 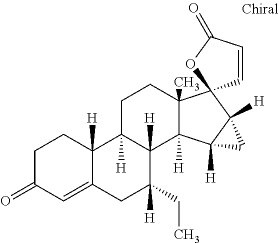 | 200 | 8.36 | 0.8 | 60 | 2.2 | 10.45 |
| 16A | 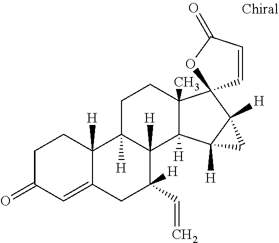 | 85 | 3.76 | 2.0 | 39 | 2.6 | 1.88 |
| 16B | 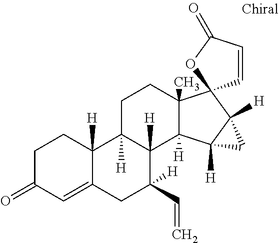 | | | | | | |
| 17A | 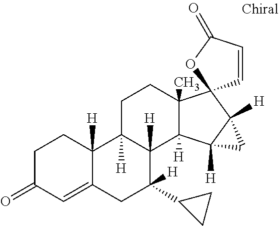 | 280 | 12.24 | 2.6 | 77 | 5.2 | 4.71 |
| 17B | 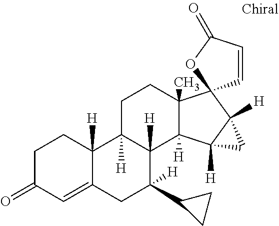 | | | | | | |
| 18 | 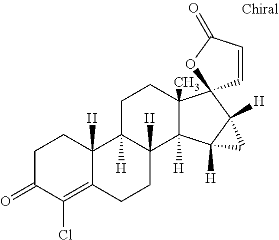 | 400 | 17.24 | 35.0 | 140 | 7.3 | 0.49 |
| 19 | 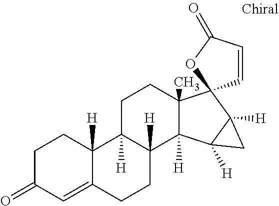 | 25 | 1.70 | 187.0 | 52 | 6.4 | 0.01 |
| 20 | 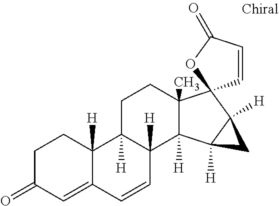 | | | | | | |
| 21 | 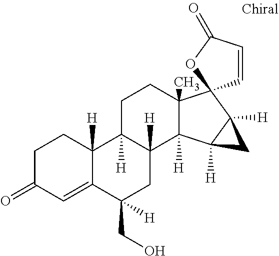 | 4800 | 159.27 | | 1000 | 1000.0 | |
| 22 | 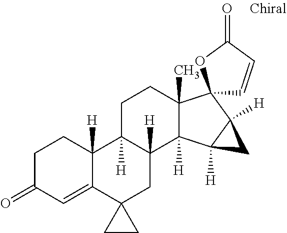 | 30 | 1.04 | 0.8 | | 2.0 | 1.30 |
| 23A | 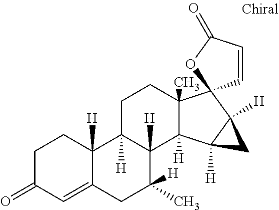 | 120 | 3.79 | 1.9 | 81 | 4.2 | 1.99 |
| 24A | 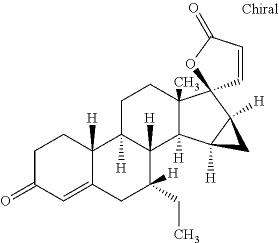 | 1300 | 41.55 | 1.9 | 160 | 8.1 | 21.87 |
| 25A | 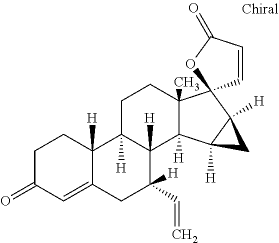 | 150 | 6.14 | 9.4 | 37 | 1.4 | 0.65 |
| 27 | 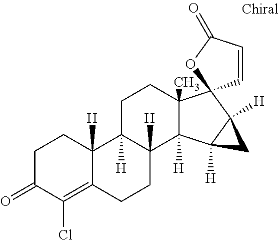 | 2900 | 101.93 | 47.0 | 810 | 36.4 | 2.17 |

TABLE 2
Values for in vitro transactivation

| | | In vitro transactivation | | | | | | | |
|---|---|---|---|---|---|---|---|---|---|
| | | Progesterone | | Mineralocorticoid | | | | | |
| | | receptor | | receptor | | Androgen receptor | | | |
| | | A- | A- | An- | An- | A- | A- | An- | An- |
| | | gonism | gonism | tagonism | tagonism | gonism | gonism | tagonism | tagonism |
| | | EC50 | efficacy | IC50 | efficacy | EC50 | efficacy | IC50 | efficacy |
| Ex. | Structure | [nM] | [%] | [nM] | [%] | [nM] | [%] | [nM] | [%] |
| A | 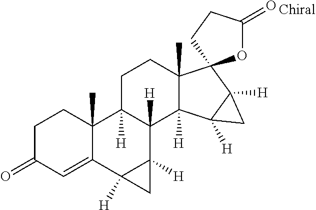 | 88 | 72.2 | 3.3 | 64.1 | 112.5 | 24.26 | 27 | 54.58 |
| 1 | 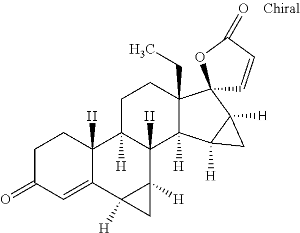 | 1.5 | 62.8 | 10 | 79.5 | 100 | 46.3 | 1000 | 5 |
| 2 | 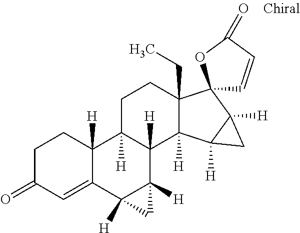 | | | 230 | 111.9 | 130 | 32.6 | 1000 | 5 |
| 3 | 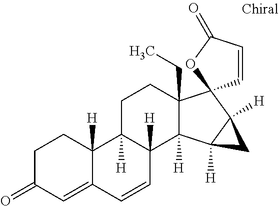 | 1.4 | 34.2 | 22 | 97.6 | 120 | 25.39 | 1000 | 5 |
| 4A | 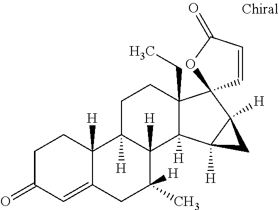 | 39.0 | 71.1 | 3 | 99.9 | 3 | 97.55 | 1000 | 5 |
| 5A | 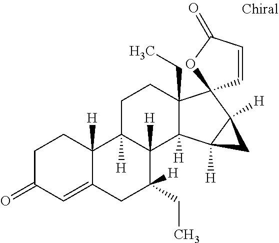 | 99.0 | 35.7 | | | 8.3 | 73 | 1000 | 5 |
| 6A | 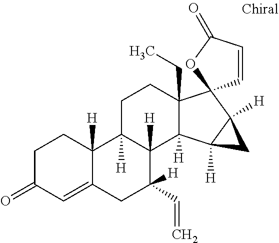 | 140.0 | 35.2 | 9 | 101.8 | 0.98 | 117.61 | 1000 | 5 |
| 7A | 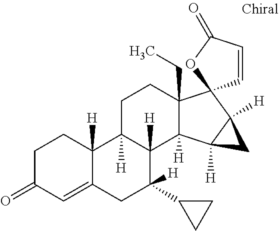 | 1000.0 | 5.0 | 77 | 101.9 | 33 | 62.57 | 1000 | 5 |
| 7B | 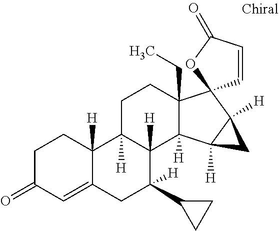 | | | | | | | | |
| 8 | 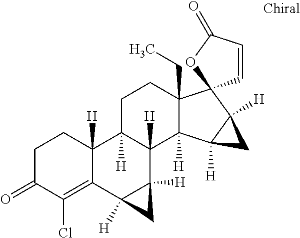 | 1000.0 | 9.8 | 960 | 31.1 | 250 | 32.89 | 130 | 22.88 |
| 9 | 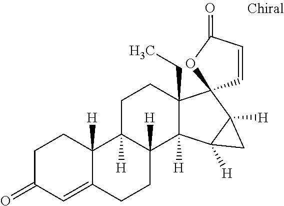 | 1.6 | 56.0 | 3 | 27.8 | 4.7 | 96.19 | 1000 | 5 |
| 10 | 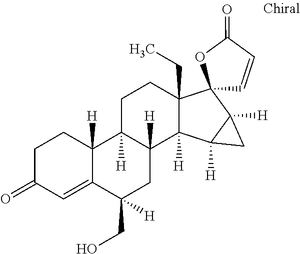 | inactive | | 110 | 92.7 | 130 | 17.87 | 160 | 15.58 |
| 12 | 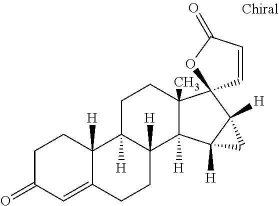 | 0.4 | 75.5 | 100 | 71.4 | 130 | 14.86 | 86 | 61.71 |
| 13 | 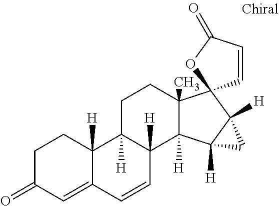 | 150.0 | 37.1 | 12 | 116.2 | 120 | 14.39 | 100 | 47.37 |
| 14A | 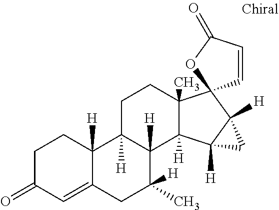 | 16.0 | 86.3 | 3 | 83.0 | 13 | 41.82 | 1.5 | 20.98 |
| 15A | 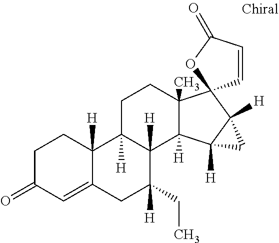 | 92.0 | 39.5 | 12 | 112.3 | 11 | 49.42 | 1000 | 5 |
| 16A | 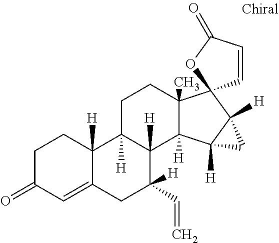 | 140.0 | 59.3 | 95 | 103.1 | 12 | 62.56 | 1000 | 5 |
| 16B | 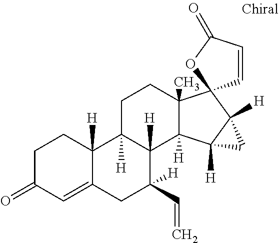 | | | | | | | | |
| 17A | 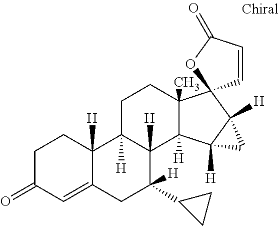 | 970.0 | 71.8 | 89 | 114.8 | 23 | 40.77 | 11 | 33.72 |
| 17B | 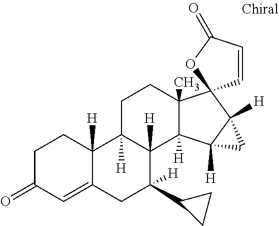 | | | | | | | | |
| 18 | 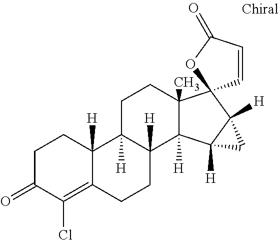 | 810.0 | 25.7 | 830 | 37.3 | 12 | 71.47 | 1000 | 5 |
| 19 | 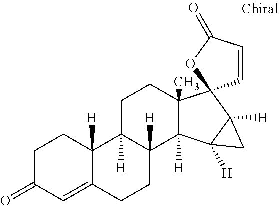 | | | 62 | 92.8 | 5.3 | 87.5 | 1000 | 5 |
| 20 | 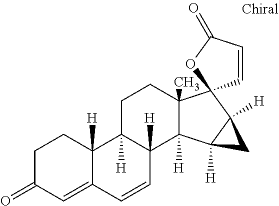 | | | | | | | | |
| 21 | 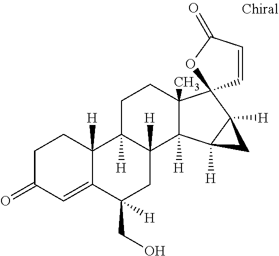 | 780.0 | 21.0 | 160 | 68.0 | 1000 | 5 | 500 | 15.68 |
| 22 | 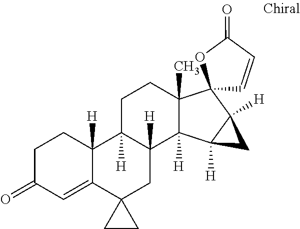 | 1.1 | 126.3 | 49 | 91.2 | 1.7 | 98.61 | 1000 | 5 |
| 23A | 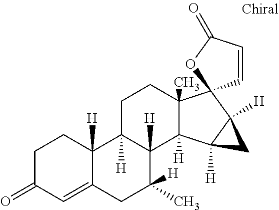 | 21.0 | 140.4 | 290 | 105.3 | 19 | 63.59 | 1000 | 5 |
| 24A | 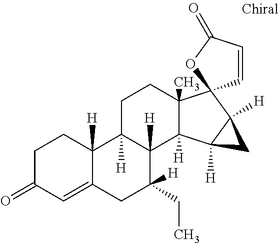 | 140.0 | 42.7 | 160 | 81.1 | 52 | 36.79 | 11 | 22.48 |
| 25A | 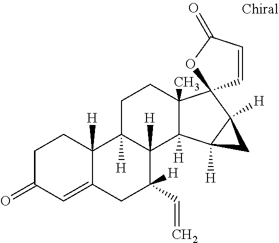 | 560.0 | 46.5 | 910 | 84.1 | 1.83 | 73.32 | 1000 | 5 |
| 27 | 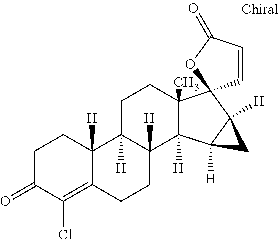 | 330.0 | 49.6 | 1000 | 5.0 | 69.5 | 66.94 | 1000 | 5 |

1. A 15,16-Methylene-17-hydroxy-19-nor-21-carboxylic acid-steroid γ-lactone derivative with the general chemical formula I: 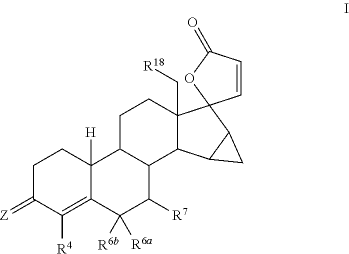
in which
Z is selected from the group comprising oxygen, two hydrogen atoms, NOR′ and NNHSO₂R′, in which R′ is hydrogen, C₁-C₁₀-alkyl, aryl or C₇-C₂₀-aralkyl,
R⁴ is selected from the group comprising hydrogen and halogen,
furthermore either:
R^{6a}, R^{6b} in each case independently of one another, are selected from the group comprising hydrogen, C₁-C₁₀-alkyl, C₂-C₁₀-alkenyl and C₂-C₁₀-alkynyl, or together form methylene or 1,2-ethanediyl and
R⁷ is selected from the group comprising hydrogen, C₁-C₁₀-alkyl, C₃-C₆-cycloalkyl, C₂-C₁₀-alkenyl and C₂-C₁₀-alkynyl,
or:
R^{6a}, R⁷ together form an oxygen atom or methylene or drop out with formation of a double bond between C⁶ and C⁷ and
R^{6b} is selected from the group comprising hydrogen, C₁-C₁₀-alkyl, C₂-C₁₀-alkenyl and C₂-C₁₀-alkynyl and
R¹⁸ is hydrogen or C₁-C₃-alkyl,
and their solvates, hydrates, stereoisomers and salts.

2. The 15,16-Methylene-17-hydroxy-19-nor-21-carboxylic acid-steroid γ-lactone derivative as claimed in claim 1, characterized in that Z is selected from the group comprising oxygen, NOR′ and NNHSO₂R′.

3. The 15,16-Methylene-17-hydroxy-19-nor-21-carboxylic acid-steroid γ-lactone derivative as claimed in claim 1, characterized in that Z stands for oxygen.

4. The 15,16-Methylene-17-hydroxy-19-nor-21-carboxylic acid-steroid γ-lactone derivative as claimed in claim 1, characterized in that R⁴ is hydrogen or chlorine.

5. The 15,16-Methylene-17-hydroxy-19-nor-21-carboxylic acid-steroid γ-lactone derivative as claimed in claim 1, characterized in that R^{6a}, R⁶⁵ together form 1,2-ethanediyl or are each hydrogen.

6. The 15,16-Methylene-17-hydroxy-19-nor-21-carboxylic acid-steroid γ-lactone derivative as claimed in claim 1, characterized in that R⁷ is selected from the group comprising hydrogen, methyl, ethyl and vinyl.

7. The 15,16-Methylene-17-hydroxy-19-nor-21-carboxylic acid-steroid γ-lactone derivative as claimed in claim 1, characterized in that R^{6a}, R⁷ together form methylene.

8. The 15,16-Methylene-17-hydroxy-19-nor-21-carboxylic acid-steroid γ-lactone derivative as claimed in claim 1, characterized in that R^{6a} and R⁷ drop out with formation of a double bond between C⁶ and C⁷.

9. The 15,16-Methylene-17-hydroxy-19-nor-21-carboxylic acid-steroid γ-lactone derivative as claimed in claim 1, characterized in that R¹⁸ is hydrogen or methyl.

10. The 15,16-Methylene-17-hydroxy-19-nor-21-carboxylic acid-steroid γ-lactone derivative as claimed in claim 1, selected from the group
17β-Hydroxy-15α,16α-methylene-19-nor-17α-pregna-4,20(Z)-dien-3-one-21-carboxylic acid γ-lactone
17β-Hydroxy-15β,16β-methylene-19-nor-17α-pregna-4,20(Z)-dien-3-one-21-carboxylic acid γ-lactone
17β-Hydroxy-7β-methyl-15α,16α-methylene-19-nor-17α-pregna-4,20(Z)-dien-3-one-21-carboxylic acid γ-lactone
17β-Hydroxy-7α-methyl-15β,16β-methylene-19-nor-17α-pregna-4,20(Z)-dien-3-one-21-carboxylic acid γ-lactone
17β-Hydroxy-7β-methyl-15β,16β-methylene-19-nor-17α-pregna-4,20(Z)-dien-3-one-21-carboxylic acid γ-lactone
17β-Hydroxy-7α-ethyl-15α,16α-methylene-19-nor-17α-pregna-4,20(Z)-dien-3-one-21-carboxylic acid γ-lactone
17β-Hydroxy-7β-ethyl-15α,16α-methylene-19-nor-17α-pregna-4,20(Z)-dien-3-one-21-carboxylic acid γ-lactone
7α-Ethyl-17β-hydroxy-15β,16β-methylene-19-nor-17α-pregna-4,20(Z)-dien-3-one-21-carboxylic acid γ-lactone
7β-Ethyl-17β-hydroxy-15β,16β-methylene-19-nor-17α-pregna-4,20(Z)-dien-3-one-21-carboxylic acid γ-lactone
17β-Hydroxy-7α-vinyl-15α,16α-methylene-19-nor-17α-pregna-4,20(Z)-dien-3-one-21-carboxylic acid γ-lactone
17β-Hydroxy-7β-vinyl-15α,16α-methylene-19-nor-17α-pregna-4,20(Z)-dien-3-one-21-carboxylic acid γ-lactone
17β-Hydroxy-15β,16β-methylene-7α-vinyl-19-nor-17α-pregna-4,20(Z)-dien-3-one-21-carboxylic acid γ-lactone
17β-Hydroxy-15β,16β-methylene-7β-vinyl-19-nor-17α-pregna-4,20(Z)-dien-3-one-21-carboxylic acid γ-lactone
17β-Hydroxy-7α-cyclopropyl-15α,16α-methylene-19-nor-17α-pregna-4,20(Z)-dien-3-one-21-carboxylic acid γ-lactone
17β-Hydroxy-4-cyclopropyl-15α,16α-methylene-19-nor-17α-pregna-4,20(Z)-dien-3-one-21-carboxylic acid γ-lactone
7α-Cyclopropyl-17β-hydroxy-14,16β-methylene-19-nor-17α-pregna-4,20(Z)-dien-3-one-21-carboxylic acid γ-lactone
17β-Hydroxy-7β-cyclopropyl-15β,16β-methylene-19-nor-17α-pregna-4,20(Z)-dien-3-one-21-carboxylic acid γ-lactone
17β-Hydroxy-6-methylene-15α,16α-methylene-19-nor-17α-pregna-4,20(Z)-dien-3-one-21-carboxylic acid γ-lactone
17β-Hydroxy-6-methylene-15β,16β-methylene-19-nor-17α-pregna-4,20(Z)-dien-3-one-21-carboxylic acid γ-lactone
17β-Hydroxy-6α-hydroxymethylene-15α,16α-methylene-19-nor-17α-pregna-4,20(Z)-dien-3-one-21-carboxylic acid γ-lactone
17β-Hydroxy-6β-hydroxymethylene-15α,16α-methylene-19-nor-17α-pregna-4,20(Z)-dien-3-one-21-carboxylic acid γ-lactone
17β-Hydroxy-6α-hydroxymethylene-141,163-methylene-19-nor-17α-pregna-4,20(Z)-dien-3-one-21-carboxylic acid γ-lactone
17β-Hydroxy-6β-hydroxymethylene-15β,16β-methylene-19-nor-17α-pregna-4,20(Z)-dien-3-one-21-carboxylic acid γ-lactone
6,6-(1,2-Ethanediyl)-17β-hydroxy-15α,16α-methylene-19-nor-17α-pregna-4,20(Z)-dien-3-one-21-carboxylic acid γ-lactone
6,6-(1,2-Ethanediyl)-17β-hydroxy-15β,16β-methylene-19-nor-17α-pregna-4,20(Z)-dien-3-one-21-carboxylic acid γ-lactone
17β-Hydroxy-6α,7α;15α,16α-bismethylene-19-nor-17α-pregna-4,20(Z)-dien-3-one-21-carboxylic acid γ-lactone
17β-Hydroxy-6β,7β;15α,16α-bismethylene-19-nor-17α-pregna-4,20(Z)-dien-3-one-21-carboxylic acid γ-lactone
17β-Hydroxy-6α,7α; 15β,16β-bismethylene-19-nor-17α-pregna-4,20(Z)-dien-3-one-21-carboxylic acid γ-lactone
17β-Hydroxy-6β,7β;15β,16β-bismethylene-19-nor-17α-pregna-4,20(Z)-dien-3-one-21-carboxylic acid γ-lactone
17β-Hydroxy-15α,16α-methylene-19-nor-17α-pregna-4,6,20(Z)-trien-3-one-21-carboxylic acid γ-lactone
17β-Hydroxy-15β,16β-methylene-19-nor-17α-pregna-4,6,20(Z)-trien-3-one-21-carboxylic acid γ-lactone
(E/Z)-3-(Hydroxyimino)-17β-hydroxy-15α,16α-methylene-19-nor-17α-pregna-4,20(Z)-diene-21-carboxylic acid γ-lactone
(E/Z)-3-(Hydroxyimino)-17β-hydroxy-15β,16β-methylene-19-nor-17α-pregna-4,20(Z)-diene-21-carboxylic acid γ-lactone
(E/Z)-3-(Hydroxyimino)-17β-hydroxy-7α-methyl-15α,16α-methylene-19-nor-17α-pregna-4,20(Z)-diene-21-carboxylic acid γ-lactone
(E/Z)-3-(Hydroxyimino)-17β-hydroxy-7β-methyl-15α,16α-methylene-19-nor-17α-pregna-4,20(Z)-diene-21-carboxylic acid γ-lactone
(E/Z)-3-(Hydroxyimino)-17β-hydroxy-7α-methyl-15β,16β-methylene-19-nor-17α-pregna-4,20(Z)-diene-21-carboxylic acid γ-lactone
(E/Z)-3-(Hydroxyimino)-17β-hydroxy-7β-methyl-15β,16β-methylene-19-nor-17α-pregna-4,20(Z)-diene-21-carboxylic acid γ-lactone
(E/Z)-3-(Hydroxyimino)-17β-hydroxy-7α-ethyl-15α,16α-methylene-19-nor-17α-pregna-4,20(Z)-diene-21-carboxylic acid γ-lactone
(E/Z)-3-(Hydroxyimino)-17β-hydroxy-7β-ethyl-15α,16α-methylene-19-nor-17α-pregna-4,20(Z)-diene-21-carboxylic acid γ-lactone
(E/Z)-3-(Hydroxyimino)-17β-hydroxy-7α-ethyl-15β,16β-methylene-19-nor-17α-pregna-4,20(Z)-diene-21-carboxylic acid γ-lactone
(E/Z)-3-(Hydroxyimino)-17β-hydroxy-7β-ethyl-15β,16β-methylene-19-nor-17α-pregna-4,20(Z)-diene-21-carboxylic acid γ-lactone
(E/Z)-3-(Hydroxyimino)-17β-hydroxy-7α-vinyl-15α,16α-methylene-19-nor-17α-pregna-4,20(Z)-diene-21-carboxylic acid γ-lactone
(E/Z)-3-(Hydroxyimino)-17β-hydroxy-7β-vinyl-15α,16α-methylene-19-nor-17α-pregna-4,20(Z)-diene-21-carboxylic acid γ-lactone
(E/Z)-3-(Hydroxyimino)-17β-hydroxy-7α-vinyl-15β,16β-methylene-19-nor-17α-pregna-4,20(Z)-diene-21-carboxylic acid γ-lactone
(E/Z)-3-(Hydroxyimino)-17β-hydroxy-7β-vinyl-15β,16β-methylene-19-nor-17α-pregna-4,20(Z)-diene-21-carboxylic acid γ-lactone
(E/Z)-3-(Hydroxyimino)-17β-hydroxy-7α-cyclopropyl-15α,16α-methylene-19-nor-17α-pregna-4,20(Z)-diene-21-carboxylic acid γ-lactone
(E/Z)-3-(Hydroxyimino)-17β-hydroxy-7β-cyclopropyl-15α,16α-methylene-19-nor-17α-pregna-4,20(Z)-diene-21-carboxylic acid γ-lactone
(E/Z)-3-(Hydroxyimino)-17β-hydroxy-7α-cyclopropyl-15β,16β-methylene-19-nor-17α-pregna-4,20(Z)-diene-21-carboxylic acid γ-lactone
(E/Z)-3-(Hydroxyimino)-17β-hydroxy-7β-cyclopropyl-15β,16β-methylene-19-nor-17α-pregna-4,20(Z)-diene-21-carboxylic acid γ-lactone
(E/Z)-3-(Hydroxyimino)-17β-hydroxy-6-methylene-15α,16α-methylene-19-nor-17α-pregna-4,20(Z)-diene-21-carboxylic acid γ-lactone
(E/Z)-3-(Hydroxyimino)-17β-hydroxy-6-methylene-15β,16β-methylene-19-nor-17α-pregna-4,20(Z)-diene-21-carboxylic acid γ-lactone
(E/Z)-3-(Hydroxyimino)-17β-hydroxy-6α-hydroxymethylene-15α,16α-methylene-19-nor-17α-pregna-4,20(Z)-diene-21-carboxylic acid γ-lactone
(E/Z)-3-(Hydroxyimino)-17β-hydroxy-6β-hydroxymethylene-15α,16α-methylene-19-nor-17α-pregna-4,20(Z)-diene-21-carboxylic acid γ-lactone
(E/Z)-3-(Hydroxyimino)-17β-hydroxy-6α-hydroxymethylene-1513,10-methylene-19-nor-17α-pregna-4,20(Z)-diene-21-carboxylic acid γ-lactone
(E/Z)-3-(Hydroxyimino)-17β-hydroxy-6β-hydroxymethylene-15β,16β-methylene-19-nor-17α-pregna-4,20(Z)-diene-21-carboxylic acid γ-lactone
(E/Z)-3-(Hydroxyimino)-6,6-(1,2-ethanediyl)-17β-hydroxy-15α,16α-methylene-19-nor-17α-pregna-4,20(Z)-diene-21-carboxylic acid γ-lactone
(E/Z)-3-(Hydroxyimino)-6,6-(1,2-ethanediyl)-17β-hydroxy-14,10-methylene-19-nor-17α-pregna-4,20(Z)-diene-21-carboxylic acid γ-lactone
(E/Z)-3-(Hydroxyimino)-17β-hydroxy-6α,7α;15α,16α-bismethylene-19-nor-17α-pregna-4,20(Z)-diene-21-carboxylic acid γ-lactone
(E/Z)-3-(Hydroxyimino)-17β-hydroxy-6β,7β;15α,16α-bismethylene-19-nor-17α-pregna-4,20(Z)-diene-21-carboxylic acid γ-lactone
(E/Z)-3-(Hydroxyimino)-17β-hydroxy-6α,7α;15β,16β-bismethylene-19-nor-17α-pregna-4,20(Z)-diene-21-carboxylic acid γ-lactone
(E/Z)-3-(Hydroxyimino)-17β-hydroxy-0, 13:14, 10-bismethylene-19-nor-17α-pregna-4,20(Z)-diene-21-carboxylic acid γ-lactone
(E/Z)-3-(Hydroxyimino)-17β-hydroxy-15α,16α-methylene-19-nor-17α-pregna-4,6,20(Z)-triene-21-carboxylic acid γ-lactone
(E/Z)-3-(Hydroxyimino)-17β-hydroxy-15β,16β-methylene-19-nor-17α-pregna-4,6,20(2)-triene-21-carboxylic acid γ-lactone
17β-Hydroxy-18-methyl-15α,16α-methylene-19-nor-17α-pregna-4,20(Z)-dien-3-one-21-carboxylic acid γ-lactone
17β-Hydroxy-18-methyl-15β,16β-methylene-19-nor-17α-pregna-4,20(Z)-dien-3-one-21-carboxylic acid γ-lactone
17β-Hydroxy-7α-methyl-18-methyl-15α,16α-methylene-19-nor-17α-pregna-4,20(Z)-dien-3-one-21-carboxylic acid γ-lactone
17β-Hydroxy-7β-methyl-18-methyl-15α,16α-methylene-19-nor-17α-pregna-4,20(Z)-dien-3-one-21-carboxylic acid γ-lactone
17β-Hydroxy-7α-methyl-18-methyl-15β,16β-methylene-19-nor-17α-pregna-4,20(Z)-dien-3-one-21-carboxylic acid γ-lactone
17β-Hydroxy-7β-methyl-18-methyl-15β,16β-methylene-19-nor-17α-pregna-4,20(Z)-dien-3-one-21-carboxylic acid γ-lactone
17β-Hydroxy-7α-ethyl-18-methyl-15α,16α-methylene-19-nor-17α-pregna-4,20(Z)-dien-3-one-21-carboxylic acid γ-lactone
17β-Hydroxy-7β-ethyl-18-methyl-15α,16α-methylene-19-nor-17α-pregna-4,20(Z)-dien-3-one-21-carboxylic acid γ-lactone
17β-Hydroxy-7α-ethyl-18-methyl-15β,16β-methylene-19-nor-17α-pregna-4,20(Z)-dien-3-one-21-carboxylic acid γ-lactone
17β-Hydroxy-7β-ethyl-18-methyl-15β,16β-methylene-19-nor-17α-pregna-4,20(Z)-dien-3-one-21-carboxylic acid γ-lactone
17β-Hydroxy-7α-vinyl-18-methyl-15α,16α-methylene-19-nor-17α-pregna-4,20(Z)-dien-3-one-21-carboxylic acid γ-lactone
17β-Hydroxy-7β-vinyl-18-methyl-15α,16α-methylene-19-nor-17α-pregna-4,20(Z)-dien-3-one-21-carboxylic acid γ-lactone
17β-Hydroxy-7α-vinyl-18-methyl-15β,16β-methylene-19-nor-17α-pregna-4,20(Z)-dien-3-one-21-carboxylic acid γ-lactone
17β-Hydroxy-7β-vinyl-18-methyl-15β,16β-methylene-19-nor-17α-pregna-4,20(Z)-dien-3-one-21-carboxylic acid γ-lactone
17β-Hydroxy-7α-cyclopropyl-18-methyl-15α,16α-methylene-19-nor-17α-pregna-4,20(Z)-dien-3-one-21-carboxylic acid γ-lactone
17β-Hydroxy-7β-cyclopropyl-18-methyl-15α,16α-methylene-19-nor-17α-pregna-4,20(Z)-dien-3-one-21-carboxylic acid γ-lactone
17β-Hydroxy-7α-cyclopropyl-18-methyl-15β,16β-methylene-19-nor-17α-pregna-4,20(Z)-dien-3-one-21-carboxylic acid γ-lactone
17β-Hydroxy-7β-cyclopropyl-18-methyl-15β,16β-methylene-19-nor-17α-pregna-4,20(Z)-dien-3-one-21-carboxylic acid γ-lactone
17β-Hydroxy-6-methylene-18-methyl-15α,16α-methylene-19-nor-17α-pregna-4,20(Z)-dien-3-one-21-carboxylic acid γ-lactone
17β-Hydroxy-6-methylene-18-methyl-15β,16β-methylene-19-nor-17α-pregna-4,20(Z)-dien-3-one-21-carboxylic acid γ-lactone
17β-Hydroxy-6α-hydroxymethylene-18-methyl-15α,16α-methylene-19-nor-17α-pregna-4,20(Z)-dien-3-one-21-carboxylic acid γ-lactone
17β-Hydroxy-6β-hydroxymethylene-18-methyl-15α,16α-methylene-19-nor-17α-pregna-4,20(Z)-dien-3-one-21-carboxylic acid γ-lactone
17β-Hydroxy-6α-hydroxymethylene-18-methyl-15β,16β-methylene-19-nor-17α-pregna-4,20(Z)-dien-3-one-21-carboxylic acid γ-lactone
17β-Hydroxy-9-hydroxymethylene-18-methyl-15β,16β-methylene-19-nor-17α-pregna-4,20(Z)-dien-3-one-21-carboxylic acid γ-lactone
6,6-(1,2-ethanediyl)-17β-Hydroxy-18-methyl-15α,16α-methylene-19-nor-17α-pregna-4,20(Z)-dien-3-one-21-carboxylic acid γ-lactone
6,6-(1,2-ethanediyl)-17β-Hydroxy-18-methyl-15β,16β-methylene-19-nor-17α-pregna-4,20(Z)-dien-3-one-21-carboxylic acid γ-lactone
17β-Hydroxy-6α,7α;15α,16α-bismethylene-18-methyl-19-nor-17α-pregna-4,20(Z)-dien-3-one-21-carboxylic acid γ-lactone
17β-Hydroxy-6β,7β,15α,16α-bismethylene-18-methyl-19-nor-17α-pregna-4,20(Z)-dien-3-one-21-carboxylic acid γ-lactone
17β-Hydroxy-6α,7α;15β,16β-bismethylene-18-methyl-19-nor-17α-pregna-4,20(Z)-dien-3-one-21-carboxylic acid γ-lactone
17β-Hydroxy-6β,7β;15β,16β-bismethylene-18-methyl-19-nor-17α-pregna-4,20(Z)-dien-3-one-21-carboxylic acid γ-lactone
17β-Hydroxy-18-methyl-15α,16α-methylene-19-nor-17α-pregna-4,6,20(Z)-trien-3-one-21-carboxylic acid γ-lactone
17β-Hydroxy-18-methyl-15β,16β-methylene-19-nor-17α-pregna-4,6,20(Z)-trien-3-one-21-carboxylic acid γ-lactone
(E/Z)-3-(Hydroxyimino)-17β-hydroxy-18-methyl-15α,16α-methylene-19-nor-17α-pregna-4,20(Z)-diene-21-carboxylic acid γ-lactone
(E/Z)-3-(Hydroxyimino)-17β-hydroxy-18-methyl-15β,16β-methylene-19-nor-17α-pregna-4,20(Z)-diene-21-carboxylic acid γ-lactone
(E/Z)-3-(Hydroxyimino)-17β-hydroxy-7α-methyl-18-methyl-15α,16α-methylene-19-nor-17α-pregna-4,20(Z)-diene-21-carboxylic acid γ-lactone
(E/Z)-3-(Hydroxyimino)-17β-hydroxy-7β-methyl-18-methyl-15α,16α-methylene-19-nor-17α-pregna-4,20(Z)-diene-21-carboxylic acid γ-lactone
(E/Z)-3-(Hydroxyimino)-17β-hydroxy-7α-methyl-18-methyl-15β,16β-methylene-19-nor-17α-pregna-4,20(Z)-diene-21-carboxylic acid γ-lactone
(E/Z)-3-(Hydroxyimino)-141-hydroxy-7β-methyl-18-methyl-15β,16β-methylene-19-nor-17α-pregna-4,20(Z)-diene-21-carboxylic acid γ-lactone
(E/Z)-3-(Hydroxyimino)-17β-hydroxy-7α-ethyl-18-methyl-15α,16α-methylene-19-nor-17α-pregna-4,20(Z)-diene-21-carboxylic acid γ-lactone
(E/Z)-3-(Hydroxyimino)-17β-hydroxy-7β-ethyl-18-methyl-15α,16α-methylene-19-nor-17α-pregna-4,20(Z)-diene-21-carboxylic acid γ-lactone
(E/Z)-3-(Hydroxyimino)-1711-hydroxy-7α-ethyl-18-methyl-15β,16β-methylene-19-nor-17α-pregna-4,20(Z)-diene-21-carboxylic acid γ-lactone
(E/Z)-3-(Hydroxyimino)-17β-hydroxy-7β-ethyl-18-methyl-15β,16β-methylene-19-nor-17α-pregna-4,20(Z)-diene-21-carboxylic acid γ-lactone
(E/Z)-3-(Hydroxyimino)-17β-hydroxy-7α-vinyl-18-methyl-15α,16α-methylene-19-nor-17α-pregna-4,20(Z)-diene-21-carboxylic acid γ-lactone
(E/Z)-3-(Hydroxyimino)-17β-hydroxy-7β-vinyl-18-methyl-15α,16α-methylene-19-nor-17α-pregna-4,20(Z)-diene-21-carboxylic acid γ-lactone
(E/Z)-3-(Hydroxyimino)-17β-hydroxy-7α-vinyl-18-methyl-15β,16β-methylene-19-nor-17α-pregna-4,20(Z)-diene-21-carboxylic acid γ-lactone
(E/Z)-3-(Hydroxyimino)-17β-hydroxy-7β-vinyl-18-methyl-15β,16β-methylene-19-nor-17α-pregna-4,20(Z)-diene-21-carboxylic acid γ-lactone
(E/Z)-3-(Hydroxyimino)-17β-hydroxy-7α-cyclopropyl-18-methyl-15α,16α-methylene-19-nor-17α-pregna-4,20(Z)-diene-21-carboxylic acid γ-lactone
(E/Z)-3-(Hydroxyimino)-17β-hydroxy-7β-cyclopropyl-18-methyl-15α,16α-methylene-19-nor-17α-pregna-4,20(Z)-diene-21-carboxylic acid γ-lactone
(E/Z)-3-(Hydroxyimino)-17β-hydroxy-7α-cyclopropyl-18-methyl-15β,16β-methylene-19-nor-17α-pregna-4,20(Z)-diene-21-carboxylic acid γ-lactone
(E/Z)-3-(Hydroxyimino)-17β-hydroxy-7β-cyclopropyl-18-methyl-15β,16β-methylene-19-nor-17α-pregna-4,20(Z)-diene-21-carboxylic acid γ-lactone
(E/Z)-3-(Hydroxyimino)-17β-hydroxy-6-methylene-18-methyl-15α,16α-methylene-19-nor-17α-pregna-4,20(Z)-diene-21-carboxylic acid γ-lactone
(E/Z)-3-(Hydroxyimino)-17β-hydroxy-6-methylene-18-methyl-15β,16β-methylene-19-nor-17α-pregna-4,20(Z)-diene-21-carboxylic acid γ-lactone
(E/Z)-3-(Hydroxyimino)-17β-hydroxy-6α-hydroxymethylene-18-methyl-15α,16α-methylene-19-nor-17α-pregna-4,20(Z)-diene-21-carboxylic acid γ-lactone
(E/Z)-3-(Hydroxyimino)-17β-hydroxy-6β-hydroxymethylene-18-methyl-15α,16α-methylene-19-nor-17α-pregna-4,20(Z)-diene-21-carboxylic acid γ-lactone
(E/Z)-3-(Hydroxyimino)-17β-hydroxy-6α-hydroxymethylene-18-methyl-15β,16β-methylene-19-nor-17α-pregna-4,20(Z)-diene-21-carboxylic acid γ-lactone
(E/Z)-3-(Hydroxyimino)-17β-hydroxy-6β-hydroxymethylene-18-methyl-15β,16β-methylene-19-nor-17α-pregna-4,20(Z)-diene-21-carboxylic acid γ-lactone
(E/Z)-3-(Hydroxyimino)-6,6-(1,2-ethanediyl)-17β-hydroxy-18-methyl-15α,16α-methylene-19-nor-17α-pregna-4,20(Z)-diene-21-carboxylic acid γ-lactone
(E/Z)-3-(Hydroxyimino)-6,6-(1,2-ethanediyl)-17β-hydroxy-18-methyl-15β,16β-methylene-19-nor-17α-pregna-4,20(Z)-diene-21-carboxylic acid γ-lactone
(E/Z)-3-(Hydroxyimino)-17β-hydroxy-6α,7α;15α,16α-bismethylene-18-methyl-19-nor-17α-pregna-4,20(Z)-diene-21-carboxylic acid γ-lactone
(E/Z)-3-(Hydroxyimino)-17β-hydroxy-6β,7β;15α,16α-bismethylene-18-methyl-19-nor-17α-pregna-4,20(Z)-diene-21-carboxylic acid γ-lactone
(E/Z)-3-(Hydroxyimino)-17β-hydroxy-6α,7α; 15β,16β-bismethylene-18-methyl-19-nor-17α-pregna-4,20(Z)-diene-21-carboxylic acid γ-lactone
(E/Z)-3-(Hydroxyimino)-17β-hydroxy-6β,7β; 15β,16β-bismethylene-18-methyl-19-nor-17α-pregna-4,20(Z)-diene-21-carboxylic acid γ-lactone
(E/Z)-3-(Hydroxyimino)-17β-hydroxy-18-methyl-15α,16α-methylene-19-nor-17α-pregna-4,6,20(Z)-triene-21-carboxylic acid γ-lactone
(E/Z)-3-(Hydroxyimino)-17β-hydroxy-18-methyl-15β,16β-methylene-19-nor-17α-pregna-4,6,20(Z)-triene-21-carboxylic acid γ-lactone
6β,7β; 15β,16β-Bismethylene-4-chloro-17β-hydroxy-18-methyl-19-nor-17α-pregna-4,20(Z)-dien-3-one-21-carboxylic acid γ-lactone
4-Chloro-17β-hydroxy-15α,16α-methylene-19-nor-17α-pregna-4,20(Z)-dien-3-one-21-carboxylic acid γ-lactone
4-Chloro-17β-hydroxy-15β,16β-methylene-19-nor-17α-pregna-4,20(Z)-dien-3-one-21-carboxylic acid γ-lactone

11. The 15,16-Methylene-17-hydroxy-19-nor-21-carboxylic acid-steroid γ-lactone derivative as claimed in claim 1 for oral contraception and for the treatment of pre-, peri- and postmenopausal complaints.

12. A method of using the 15,16-methylene-17-hydroxy-19-nor-21 carboxylic acid-steroid γ-lactone derivative as claimed in claim 1 for comprising producing a medicinal product for oral contraception and for the treatment of pre-, peri- and postmenopausal complaints with said derivative.

13. The use as claimed in claim 12, characterized in that the medicinal product has progestational, antimineralocorticoid and neutral to slightly androgenic action.

14. A medicinal product containing at least one 15,16-methylene-17-hydroxy-19-nor-21-carboxylic acid-steroid γ-lactone derivative as claimed in claim 1 and at least one suitable pharmaceutically harmless additive.

15. The medicinal product as claimed in claim 14, additionally containing at least one estrogen.

16. The medicinal product as claimed in claim 15, characterized in that the estrogen is ethinylestradiol.

17. The medicinal product as claimed in claim 15, characterized in that the natural estrogen is estradiol valerate.

18. The medicinal product as claimed in claim 15, characterized in that the estrogen is a natural estrogen.

19. The medicinal product as claimed in claim 18, characterized in that the natural estrogen is estradiol.

20. The medicinal product as claimed in claim 18, characterized in that the natural estrogen is a conjugated estrogen.

21. A method of using the 15,16-methylene-17-hydroxy-19-nor-21-carboxylic acid steroid γ-lactone derivative as claimed in claim 1 comprising producing a medicinal product for intrauterine use with said derivative.

22. The use as claimed in claim 21 for the production of an intrauterine system (IUS).

23. Medicinal containing at least one 15,16-methylene-17-hydroxy-19-nor-21-carboxylic acid steroid γ-lactone derivative according to claim 1 and at least one suitable pharmaceutically harmless additive, for intrauterine use.

24. The medicinal product as claimed in claim 23, characterized in that it is an intrauterine system.
